(19) United States
(12) Patent Application Publication
(10) Pub. No.: US 2023/0365619 A1
(43) Pub. Date: Nov. 16, 2023

(54) CRYSTALLINE SODIUM CHOLESTERYL SULFATE

(71) Applicant: Durect Corporation, Cupertino, CA (US)

(72) Inventors: WEIQI LIN, Emerald Hills, CA (US); STEPHAN D. PARENT, West Lafayette, IN (US); COURTNEY S. JOHNSON, West Lafayette, IN (US)

(21) Appl. No.: 18/028,451

(22) PCT Filed: Oct. 11, 2021
(86) PCT No.: PCT/US2021/054425

§ 371 Date: Mar. 24, 2023

Related U.S. Application Data
(60) Provisional application No. 63/090,608, filed on Oct. 12, 2020.

(51) Int. Cl. C07J 31/00

(52) U.S. Cl. CPC C07J 31/006; C07B 2200/13

(57) ABSTRACT

Crystalline forms of crystalline sodium cholesteryl sulfate are disclosed herein. Such forms include crystalline sodium cholesteryl sulfate Form B, crystalline sodium cholesteryl sulfate Form F, crystalline sodium cholesteryl sulfate Form H, crystalline sodium cholesteryl sulfate Form J, crystalline sodium cholesteryl sulfate Form K, crystalline sodium cholesteryl sulfate Form L, crystalline sodium cholesteryl sulfate Form M, and crystalline sodium cholesteryl sulfate Form N. Processes for making such crystalline forms and methods of treating disease with form are further provided.

### CROSS REFERENCE TO RELATED APPLICATIONS

This application claims priority to U.S. Provisional Application No. 63/090,608, filed Oct. 12, 2020, which is incorporated herein by reference.

### BACKGROUND

It has been shown previously that nuclear cholesterol metabolite cholesterol-3-sulfate (“sodium cholesteryl sulfate”) decreases lipid biosynthesis and increases cholesterol secretion and degradation, and may be useful for the treatment and prevention of hypercholesterolemia, hypertriglyceridemia, and conditions related to fat-accumulation and inflammation (e.g. non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, and atherosclerosis).

Cholesterol is used by the body for the manufacture and repair of cell membranes, and the synthesis of steroid hormones and vitamin D, and is transformed to bile acids in the liver. There are both exogenous and endogenous sources of cholesterol. The average American consumes about 450 mg of cholesterol each day and produces an additional 500 mg to 1,000 mg in the liver and other tissues. Another source is the 500 mg to 1,000 mg of biliary cholesterol that is secreted into the intestine daily, and about 50 percent is reabsorbed (enterohepatic circulation).

High serum lipid levels (hypercholesterolemia and hypertriglyceridemia) are associated with the accumulation of cholesterol in arterial walls, and can result in NAFLD and atherosclerosis. The plaques that characterize atherosclerosis inhibit blood flow and promote clot formation, and can ultimately cause death or severe disability via heart attacks and/or stroke. A number of therapeutic agents for the treatment of hyperlipidemia have been developed and are widely prescribed by physicians. Unfortunately, only about 35% of patients are responsive to the currently available therapies.

Non-alcoholic fatty liver disease (NAFLD) is the most common liver disease in the United States. This condition is associated with obesity, type-II adult onset diabetes, sedentary lifestyle, and diets high in fat. The earlier stage of NAFLD, fatty liver, is potentially reversible when proper treatment steps are taken. However, left unchecked, it can progress to inflammation of liver cells (non-alcoholic steatohepatitis, or NASH) which is much more difficult to treat. Without treatment, NASH can result in irreversible scarring of liver tissue (steatonecrosis), with the potential to cause cirrhosis, liver failure, and liver cancer.

Crystalline solids are generally favorable for processing, storage, and stability than non-crystalline amorphous solids, for example. However, energetics may not favor the ready formation of suitable crystalline solids and polymorphism may make creating stable crystalline solids of a particular active pharmaceutical ingredient impractical. Herein, the inventors disclose crystalline sodium cholesteryl sulfate.

### SUMMARY

In some aspects of the present disclosure, crystalline sodium cholesteryl sulfate is provided.

In other aspects of the present disclosure, stable crystalline sodium cholesteryl sulfate is provided.

In further aspects of the present disclosure, anhydrates of crystalline sodium cholesteryl sulfate are provided.

In other aspects of the present disclosure, hydrates of crystalline sodium cholesteryl sulfate are provided.

In yet additional aspects of the disclosure, solvates of crystalline sodium cholesteryl sulfate are provided.

In additional aspects of the present disclosure, crystalline sodium cholesteryl sulfate Form B, crystalline sodium cholesteryl sulfate Form F, crystalline sodium cholesteryl sulfate Form H, crystalline sodium cholesteryl sulfate Form J, crystalline sodium cholesteryl sulfate Form K, crystalline sodium cholesteryl sulfate Form L, crystalline sodium cholesteryl sulfate Form M, and crystalline sodium cholesteryl sulfate Form N are provided.

In yet additional aspects of the present disclosure, mixtures of two or more of crystalline sodium cholesteryl sulfate Form B, crystalline sodium cholesteryl sulfate Form F, crystalline sodium cholesteryl sulfate Form H, crystalline sodium cholesteryl sulfate Form J, crystalline sodium cholesteryl sulfate Form K, crystalline sodium cholesteryl sulfate Form L, crystalline sodium cholesteryl sulfate Form M, and crystalline sodium cholesteryl sulfate Form N are provided.

In other aspects of the present disclosure, methods of treating or preventing one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis, comprising administering to a patient in need thereof an effective amount of a compound or pharmaceutical composition thereof of crystalline sodium cholesteryl sulfate Form B, crystalline sodium cholesteryl sulfate Form F, crystalline sodium cholesteryl sulfate Form H, crystalline sodium cholesteryl sulfate Form J, crystalline sodium cholesteryl sulfate Form K, crystalline sodium cholesteryl sulfate Form L, crystalline sodium cholesteryl sulfate Form M, and crystalline sodium cholesteryl sulfate Form N are provided.

In further aspects of the present disclosure, pharmaceutical compositions comprising one or more of crystalline sodium cholesteryl sulfate Form B, crystalline sodium cholesteryl sulfate Form F, crystalline sodium cholesteryl sulfate Form H, crystalline sodium cholesteryl sulfate Form J, crystalline sodium cholesteryl sulfate Form K, crystalline sodium cholesteryl sulfate Form L, crystalline sodium cholesteryl sulfate Form M, and crystalline sodium cholesteryl sulfate Form N are provided and at least one pharmaceutically acceptable excipient are provided.

In additional aspects of the present disclosure, processes for making crystalline sodium cholesteryl sulfate Form B, crystalline sodium cholesteryl sulfate Form F, crystalline sodium cholesteryl sulfate Form H, crystalline sodium cholesteryl sulfate Form J, crystalline sodium cholesteryl sulfate Form K, crystalline sodium cholesteryl sulfate Form L, crystalline sodium cholesteryl sulfate Form M, and crystalline sodium cholesteryl sulfate Form N are provided.

In further aspects of the present disclosure, use of one or more of crystalline sodium cholesteryl sulfate Form B, crystalline sodium cholesteryl sulfate Form F, crystalline sodium cholesteryl sulfate Form H, crystalline sodium cholesteryl sulfate Form J, crystalline sodium cholesteryl sulfate Form K, crystalline sodium cholesteryl sulfate Form L, crystalline sodium cholesteryl sulfate Form M, or crystalline sodium cholesteryl sulfate Form N are provided, and optionally one or more pharmaceutically acceptable excipients, for treating host mammal with hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis, are provided.

In yet additional aspects of the present disclosure, crystalline sodium cholesteryl sulfate, a mixture of crystalline sodium cholesteryl sulfate of any of claims, or a pharmaceutical composition of crystalline sodium cholesteryl sulfate for use as a medicament are provided.

In still further aspects of the present disclosure, crystalline sodium cholesteryl sulfate, a mixture of crystalline sodium cholesteryl sulfate, or a pharmaceutical composition of crystalline sodium cholesteryl sulfate any of claims, for use in a method of treating or preventing one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis are provided.

### BRIEF DESCRIPTION OF THE FIGURES

FIG. 1 is a peak picked x-ray powder diffraction pattern of crystalline sodium cholesteryl sulfate Form F of the full diffractogram.

FIG. 2 is a peak-picked x-ray powder diffraction pattern of crystalline sodium cholesteryl sulfate Form F from about 4°2θ to about 29°2θ.

FIG. 3 is a Differential Scanning calorimetry (“DSC”) thermogram for crystalline sodium cholesteryl sulfate Form F.

FIG. 4 is a Dynamic Vapor Sorption (“DVS”) plot for crystalline sodium cholesteryl sulfate Form F.

FIG. 5 is a peak-picked x-ray powder diffraction pattern of crystalline sodium cholesteryl sulfate Form H of the full diffractogram.

FIG. 6 is a peak-picked x-ray powder diffraction pattern of crystalline sodium cholesteryl sulfate Form H from about 4°2θ to about 29°2θ.

FIG. 7 is a DSC Thermogram of crystalline sodium cholesteryl sulfate Form H.

FIG. 8 is an x-ray powder diffraction pattern of crystalline sodium cholesteryl sulfate Form J of the full diffractogram.

FIG. 9 is a DSC thermogram for crystalline sodium cholesteryl sulfate Form J.

FIG. 10 is an x-ray powder diffraction pattern of crystalline sodium cholesteryl sulfate Form K of the full diffractogram.

FIG. 11 is the indexing solution for crystalline sodium cholesteryl sulfate Form K.

FIG. 12 is a DSC thermogram of crystalline sodium cholesteryl sulfate Form K.

FIG. 13 is a peak-picked x-ray powder diffraction pattern of crystalline sodium cholesteryl sulfate Form L of the full diffractogram.

FIG. 14 is a peak-picked x-ray powder diffraction pattern of crystalline sodium cholesteryl sulfate Form L from about 4°2θ to about 29°2θ.

FIG. 15 is a DSC thermogram of crystalline sodium cholesteryl sulfate Form L.

FIG. 16 is an x-ray powder diffraction pattern of crystalline sodium cholesteryl sulfate Form M of the full diffractogram.

FIG. 17 is the indexing solution for crystalline sodium cholesteryl sulfate Form M.

FIG. 18 is a DSC thermogram for crystalline sodium cholesteryl sulfate Form M.

FIG. 19 is an x-ray powder diffraction pattern of crystalline sodium cholesteryl sulfate Form N of the full diffractogram.

FIG. 20 is a DSC thermogram of crystalline sodium cholesteryl sulfate Form N.

FIG. 21 is an x-ray powder diffraction pattern of crystalline sodium cholesteryl sulfate Form B.

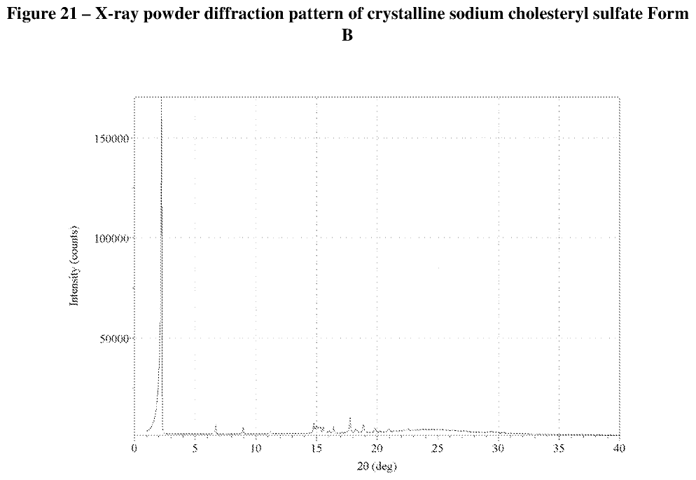

### DETAILED DESCRIPTION

Crystalline sodium cholesteryl sulfate is readily analyzed by x-ray powder diffraction. An x-ray powder diffraction pattern is an x-y graph with °2θ (diffraction angle) on the x-axis and intensity on the y-axis. The pattern contains peaks which may be used to characterize crystalline sodium cholesteryl sulfate. The peaks are usually represented and referred to by their position on the x-axis. Unless otherwise specified, peaks are referred to by their position on the x-axis and not their y-axis intensity.

The data from x-ray powder diffraction may be used in multiple ways to characterize crystalline forms. For example, the entire x-ray powder diffraction pattern output from a diffractometer may be used to characterize crystalline sodium cholesteryl sulfate. A smaller subset of such data, however, may also be, and typically is, suitable for characterizing crystalline sodium cholesteryl sulfate. For example, a collection of one or more peaks from such a pattern may be used to characterize crystalline sodium cholesteryl sulfate. In the present application, all reported peak values are in °2θ with Cu-Kα radiation as set forth in Example 10. Indeed, often even a single x-ray powder diffraction peak may be used to characterize such a crystalline form. When crystalline sodium cholesteryl sulfate herein is characterized by “one or more peaks” of an x-ray powder diffraction pattern and such peaks are listed, what is generally meant is that any combination of the peaks listed may be used to characterize crystalline sodium cholesteryl sulfate. Further, the fact that other peaks are present in the x-ray powder diffraction pattern, generally does not negate or otherwise limit that characterization.

In addition to the variability in peak intensity, there may also be variability in the position of peaks on the x-axis. This variability can, however, typically be accounted for when reporting the positions of peaks for purposes of characterization. Such variability in the position of peaks along the x-axis may derive from several sources (e.g., sample preparation, particle size, moisture content, solvent content, instrument parameters, data analysis software, and sample orientation). For example, samples of the same crystalline material prepared under different conditions may yield slightly different diffractograms, and different x-ray instruments may operate using different parameters and these may lead to slightly different diffraction patterns from the same crystalline solid.

Due to such sources of variability, it is common to recite x-ray diffraction peaks using the word “about” prior to the peak value in °2θ. For purposes of data reported herein, that value is generally ±0.2°2θ. This generally means that on a well-maintained instrument one would expect the variability in peak measurement to be ±0.2°2θ or less. X-ray powder diffraction peaks cited herein are generally reported with this variability of ±0.2°2θ unless stated otherwise and are generally intended to be reported with such a variability whenever disclosed herein whether the word “about” is present or not, unless context dictates otherwise. Depending on instrument type and calibration, for example, it is possible for an instrument's variability to less than ±0.2°2θ. Accordingly, in certain embodiments, the variability in a peak value or grouping of peak values ±0.1°2θ, or even ±0.05°2θ, rather than ±0.2°2θ.

Thermal methods are another typical technique to characterize solid forms such as salts. Different polymorphs of the same compound often have different endothermic events such as when measured by Differential Scanning calorimetry. Such events may include melting. As with any analytical technique, melting point determinations are also subject to variability. Common sources of variability, in addition to instrumental variability, are due to colligative properties such as the presence of other solid forms or other impurities within a sample whose melting point is being measured. Common variability for thermal measurements is on the order of ±1° C. and are generally intended to be reported with such a variability whenever disclosed herein whether the word “about” is present or not.

Sodium cholesteryl sulfate has the following chemical structure:

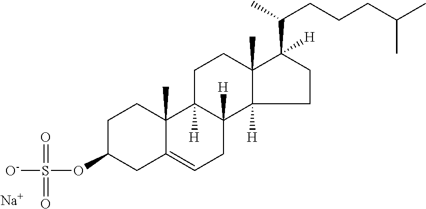

The present disclosure uses the term “Form” to identify different crystalline forms of crystalline sodium cholesteryl sulfate. The differences in the forms can be seen by structure, such as x-ray powder diffraction; properties such as hygroscopicity or thermal behaviors; and/or both. The use of the term “Form B” means crystalline sodium cholesteryl sulfate Form B. Likewise, “Form F” means crystalline sodium cholesteryl sulfate Form F, “Form H” means crystalline sodium cholesteryl sulfate Form H, “Form J” means crystalline sodium cholesteryl sulfate Form J, “Form K” means crystalline sodium cholesteryl sulfate Form K, “Form L” means crystalline sodium cholesteryl sulfate Form L, “Form M” means crystalline sodium cholesteryl sulfate Form M, and “Form N” means crystalline sodium cholesteryl sulfate Form N.

Multiple crystalline forms, including stable crystalline forms, of sodium cholesteryl sulfate are herein reported. These are crystalline sodium cholesteryl sulfate Form B, crystalline sodium cholesteryl sulfate Form F, crystalline sodium cholesteryl sulfate Form H, crystalline sodium cholesteryl sulfate Form J, crystalline sodium cholesteryl sulfate Form K, crystalline sodium cholesteryl sulfate Form L, crystalline sodium cholesteryl sulfate Form M, and crystalline sodium cholesteryl sulfate Form N are provided herein. In the present disclosure, “stable” means that the form does not readily interconvert to another form under a given set of conditions. A metastable form, can, however, so readily convert when exposed to certain conditions. Thus, a form that is stable under one set of conditions (e.g., humidity) may not be stable under another set of conditions. In many embodiments, crystalline sodium cholesteryl sulfate is provided, including stable crystalline sodium cholesteryl sulfate. The crystalline sodium cholesteryl sulfate of the present disclosure includes solvates, hydrates, and anhydrates. Solvates include alcohol solvates such as methanol, ethanol, isopropanol, and propanol solvates, for example.

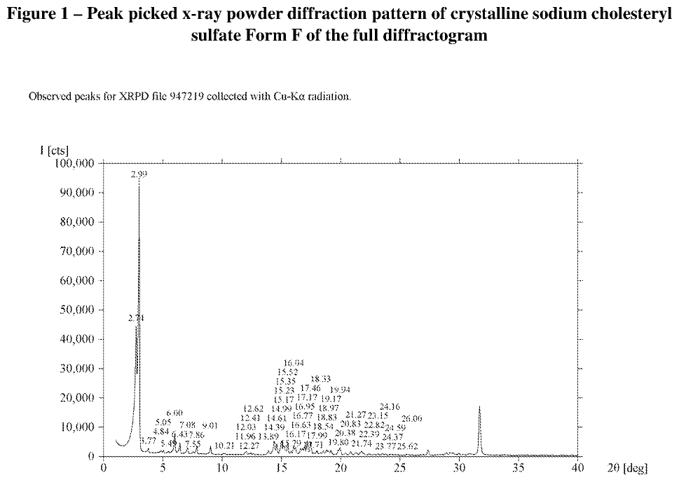

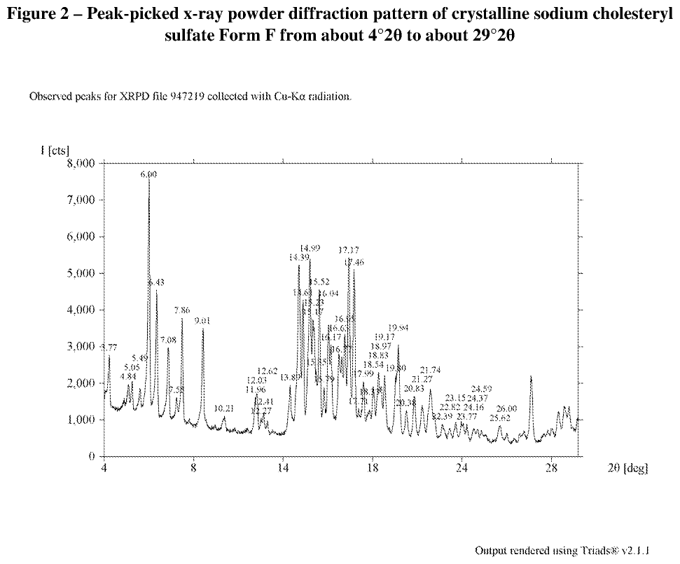

In many embodiments, crystalline sodium cholesteryl sulfate Form F is provided. Without being bound by theory, it is believed crystalline sodium cholesteryl sulfate Form F is an anhydrate. A preparation of crystalline sodium cholesteryl sulfate Form F is set forth in Example 1. An x-ray powder diffraction pattern for crystalline sodium cholesteryl sulfate Form F is set forth in FIG. 1 which also indicates specifically identified peaks. An expanded set of peaks is set forth in FIG. 2. Table 1 also shows peaks identified in FIG. 1 and FIG. 2.

TABLE 1
Observed peaks for Crystalline Sodium Cholesteryl Sulfate Form F

| °2θ | d space (Å) | Intensity (%) |
|---|---|---|
| 2.74 ± 0.20 | 32.215 ± 2.351 | 48 |
| 2.99 ± 0.20 | 29.534 ± 1.976 | 100 |
| 3.77 ± 0.20 | 23.417 ± 1.242 | 3 |
| 4.84 ± 0.20 | 18.224 ± 0.752 | 2 |
| 5.05 ± 0.20 | 17.492 ± 0.693 | 2 |
| 5.49 ± 0.20 | 16.072 ± 0.585 | 2 |
| 6.00 ± 0.20 | 14.722 ± 0.490 | 8 |
| 6.43 ± 0.20 | 13.732 ± 0.427 | 5 |
| 7.08 ± 0.20 | 12.481 ± 0.352 | 3 |
| 7.55 ± 0.20 | 11.707 ± 0.310 | 2 |
| 7.86 ± 0.20 | 11.245 ± 0.286 | 4 |
| 9.01 ± 0.20 | 9.802 ± 0.217 | 4 |
| 10.21 ± 0.20 | 8.656 ± 0.169 | 1 |
| 11.96 ± 0.20 | 7.396 ± 0.123 | 2 |
| 12.03 ± 0.20 | 7.353 ± 0.122 | 2 |
| 12.27 ± 0.20 | 7.205 ± 0.117 | 1 |
| 12.41 ± 0.20 | 7.128 ± 0.114 | 1 |
| 12.62 ± 0.20 | 7.011 ± 0.111 | 1 |
| 13.89 ± 0.20 | 6.372 ± 0.091 | 2 |
| 14.39 ± 0.20 | 6.151 ± 0.085 | 6 |
| 14.61 ± 0.20 | 6.058 ± 0.082 | 5 |
| 14.99 ± 0.20 | 5.904 ± 0.078 | 6 |
| 15.17 ± 0.20 | 5.836 ± 0.076 | 4 |
| 15.23 ± 0.20 | 5.813 ± 0.076 | 4 |
| 15.35 ± 0.20 | 5.769 ± 0.075 | 3 |
| 15.52 ± 0.20 | 5.703 ± 0.073 | 5 |
| 15.79 ± 0.20 | 5.609 ± 0.071 | 2 |
| 16.04 ± 0.20 | 5.523 ± 0.068 | 4 |
| 16.17 ± 0.20 | 5.478 ± 0.067 | 3 |
| 16.63 ± 0.20 | 5.328 ± 0.064 | 3 |
| 16.77 ± 0.20 | 5.281 ± 0.063 | 3 |
| 16.95 ± 0.20 | 5.227 ± 0.061 | 4 |
| 17.17 ± 0.20 | 5.160 ± 0.060 | 6 |
| 17.46 ± 0.20 | 5.074 ± 0.058 | 5 |
| 17.71 ± 0.20 | 5.004 ± 0.056 | 1 |
| 17.99 ± 0.20 | 4.927 ± 0.054 | 2 |
| 18.33 ± 0.20 | 4.836 ± 0.052 | 1 |
| 18.54 ± 0.20 | 4.782 ± 0.051 | 2 |
| 18.83 ± 0.20 | 4.708 ± 0.050 | 2 |
| 18.97 ± 0.20 | 4.674 ± 0.049 | 2 |
| 19.17 ± 0.20 | 4.625 ± 0.048 | 2 |
| 19.80 ± 0.20 | 4.481 ± 0.045 | 2 |
| 19.94 ± 0.20 | 4.450 ± 0.044 | 3 |
| 20.38 ± 0.20 | 4.353 ± 0.042 | 1 |
| 20.83 ± 0.20 | 4.261 ± 0.040 | 2 |
| 21.27 ± 0.20 | 4.173 ± 0.039 | 2 |
| 21.74 ± 0.20 | 4.085 ± 0.037 | 2 |
| 22.39 ± 0.20 | 3.967 ± 0.035 | 1 |
| 22.82 ± 0.20 | 3.894 ± 0.034 | 1 |
| 23.15 ± 0.20 | 3.839 ± 0.033 | 1 |
| 23.77 ± 0.20 | 3.740 ± 0.031 | 1 |
| 24.16 ± 0.20 | 3.681 ± 0.030 | 1 |
| 24.37 ± 0.20 | 3.650 ± 0.030 | 1 |
| 24.59 ± 0.20 | 3.617 ± 0.029 | 1 |
| 25.62 ± 0.20 | 3.474 ± 0.027 | 1 |
| 26.00 ± 0.20 | 3.424 ± 0.026 | 1 |

Crystalline sodium cholesteryl sulfate Form F may be characterized by various analytical techniques, including by x-ray powder diffraction. The x-ray powder diffraction pattern of crystalline sodium cholesteryl sulfate Form F, or portions thereof, may be used to identify crystalline sodium cholesteryl sulfate Form F. Crystalline sodium cholesteryl sulfate Form F contains various x-ray powder diffraction peaks which alone or together may help identify the presence of crystalline sodium cholesteryl sulfate Form F. For example, in many embodiments, crystalline sodium cholesteryl sulfate Form F may be characterized by an x-ray powder diffraction pattern with two peaks between about 1.9°2θ and about 3.3°2θ. In these and other embodiments, one of the two peaks may be a peak at about 2.7°2θ or about 3.0°2θ. In these and other embodiments, crystalline sodium cholesteryl sulfate Form F may be characterized by an x-ray powder diffraction pattern comprising two peaks between about 1.9°2θ and about 3.3°2θ where; for example, one peak may be at about 2.7°2θ and another at about 3.0°2θ and may optionally comprise one or more peaks at about 6.0°2θ and about 6.4°2θ. In other embodiments, crystalline sodium cholesteryl sulfate Form F may be characterized by an x-ray powder diffraction pattern substantially the same as that found in FIG. 1. In various other embodiments, crystalline sodium cholesteryl sulfate Form F may be characterized by an x-ray powder diffraction pattern substantially the same as that found in FIG. 2, which is a diffraction pattern of an expanded region of FIG. 1.

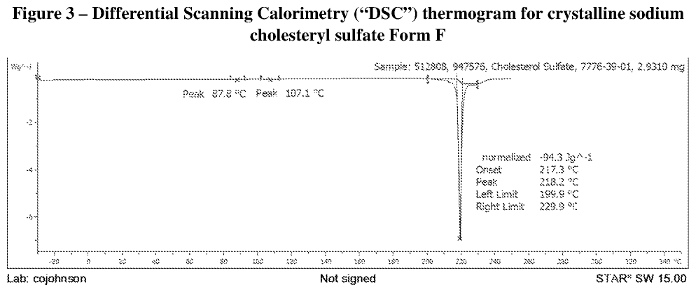

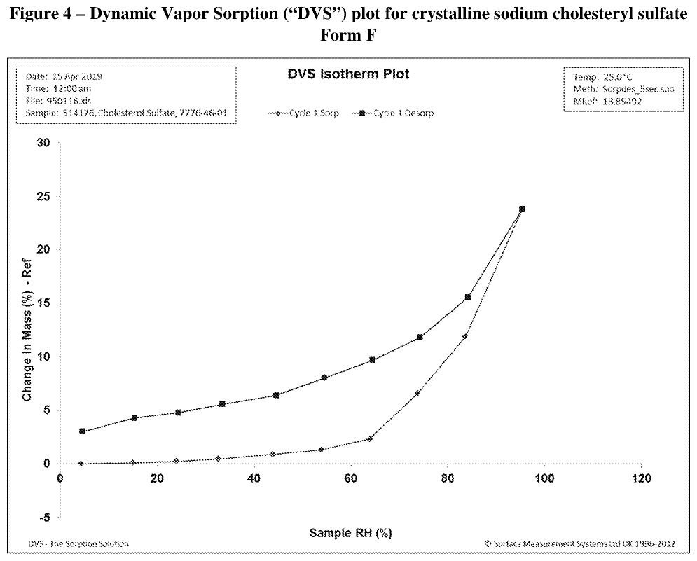

A DSC thermogram for crystalline sodium cholesteryl sulfate Form F is shown in FIG. 3. It indicates an onset of about 217° C., which may be used to characterize crystalline sodium cholesteryl sulfate Form F alone or in connection with XRPD data used to characterize crystalline sodium cholesteryl sulfate Form F. A DVS plot for crystalline sodium cholesteryl sulfate Form F is shown in FIG. 4. It indicates 23.8% weight gain on sorption and 20.8% upon desorption with little weight gain until 55% relative humidity on ramping from 5% to 95% relative humidity.

Substantially pure crystalline sodium cholesteryl sulfate Form F is further disclosed. “Substantially pure,” as described herein, generally refers to a form herein that is present without any appreciable amounts, other than potentially trace levels of other forms of crystalline sodium cholesteryl sulfate. Examples of trace levels include not more than about 10%, 5%, 2%, 1.5%, 1%, 0.5%, 0.25%, 0.1%, or less by weight in total relative to the total amount of crystalline sodium cholesteryl sulfate present.

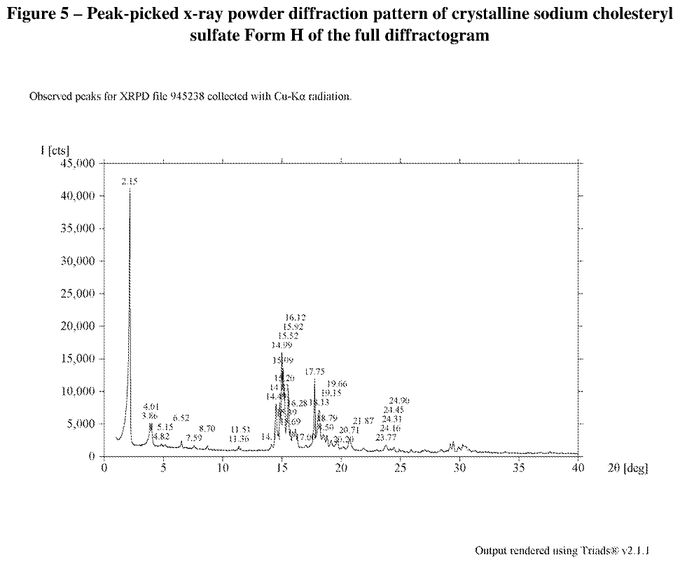

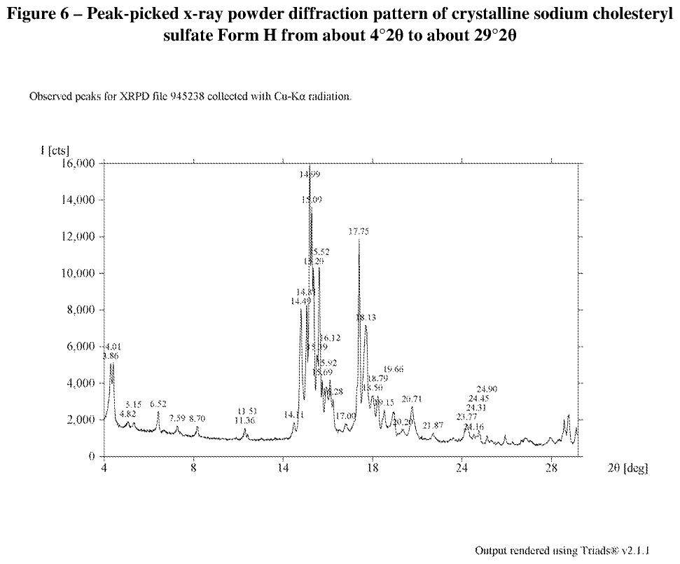

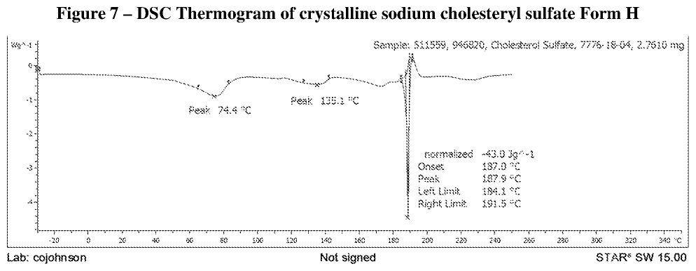

In many embodiments, crystalline sodium cholesteryl sulfate Form H is provided. Without being bound by theory, it is believed that crystalline sodium cholesteryl sulfate Form H is a hydrate, solvate, or mixture of the two. The ¹H-NMR spectrum of crystalline sodium cholesteryl sulfate Form H indicates the solvent NMP (N-methyl-2-pyrrolidone) present at a half-molar basis with respect to crystalline sodium cholesteryl sulfate Form H when crystalline sodium cholesteryl sulfate Form H is made with NMP, as in Example 3. Example 3 sets forth a preparation of crystalline sodium cholesteryl sulfate Form H. An x-ray powder diffraction pattern for crystalline sodium cholesteryl sulfate Form H is set forth in FIG. 5 with an expanded diffraction pattern in FIG. 6. Table 2 sets forth specifically identified peaks from FIG. 5 and FIG. 6. FIG. 7 is a DSC thermogram of crystalline sodium cholesteryl sulfate Form H.

Crystalline sodium cholesteryl sulfate Form H may be characterized by various analytical techniques such as x-ray powder diffraction. The x-ray powder diffraction pattern of crystalline sodium cholesteryl sulfate Form H or portions thereof may be used to characterize crystalline sodium cholesteryl sulfate Form H. Crystalline sodium cholesteryl sulfate Form H contains various x-ray powder diffraction peaks which alone or together may help characterize crystalline sodium cholesteryl sulfate Form H. For example, crystalline sodium cholesteryl sulfate Form H may be characterized by an x-ray powder diffraction pattern comprising a peak at about 2.2°2θ. The x-ray powder diffraction pattern may further comprise a peak at about 3.9°2θ or a peak at about 4.0°2θ. In these and other embodiments, crystalline sodium cholesteryl sulfate Form H may be characterized by an x-ray powder diffraction pattern comprising a peak at about 2.2°2θ and further comprising one or more peaks at about 4.8°2θ, about 5.2°2θ, or a peak at about 6.5°2θ.

TABLE 2
Observed peaks for Crystalline Sodium Cholesteryl Sulfate Form H

| °2θ | d space (Å) | Intensity (%) |
|---|---|---|
| 2.15 ± 0.20 | 40.991 ± 3.806 | 100 |
| 3.86 ± 0.20 | 22.853 ± 1.183 | 13 |
| 4.01 ± 0.20 | 22.042 ± 1.100 | 13 |
| 4.82 ± 0.20 | 18.309 ± 0.759 | 5 |
| 5.15 ± 0.20 | 17.145 ± 0.665 | 5 |
| 6.52 ± 0.20 | 13.553 ± 0.416 | 6 |
| 7.59 ± 0.20 | 11.640 ± 0.306 | 4 |
| 8.70 ± 0.20 | 10.150 ± 0.233 | 4 |
| 11.36 ± 0.20 | 7.780 ± 0.136 | 4 |
| 11.51 ± 0.20 | 7.680 ± 0.133 | 3 |
| 14.11 ± 0.20 | 6.272 ± 0.088 | 5 |
| 14.49 ± 0.20 | 6.107 ± 0.084 | 20 |
| 14.81 ± 0.20 | 5.975 ± 0.080 | 21 |
| 14.99 ± 0.20 | 5.907 ± 0.078 | 40 |
| 15.09 ± 0.20 | 5.867 ± 0.077 | 34 |
| 15.20 ± 0.20 | 5.823 ± 0.076 | 26 |
| 15.39 ± 0.20 | 5.753 ± 0.074 | 14 |
| 15.52 ± 0.20 | 5.705 ± 0.073 | 26 |
| 15.69 ± 0.20 | 5.643 ± 0.071 | 10 |
| 15.92 ± 0.20 | 5.564 ± 0.069 | 10 |
| 16.12 ± 0.20 | 5.493 ± 0.068 | 10 |
| 16.28 ± 0.20 | 5.439 ± 0.066 | 8 |
| 17.00 ± 0.20 | 5.210 ± 0.061 | 4 |
| 17.75 ± 0.20 | 4.992 ± 0.056 | 29 |
| 18.13 ± 0.20 | 4.889 ± 0.053 | 18 |
| 18.50 ± 0.20 | 4.792 ± 0.051 | 8 |
| 18.79 ± 0.20 | 4.718 ± 0.050 | 8 |
| 19.15 ± 0.20 | 4.631 ± 0.048 | 6 |
| 19.66 ± 0.20 | 4.512 ± 0.045 | 6 |
| 20.20 ± 0.20 | 4.393 ± 0.043 | 4 |
| 20.71 ± 0.20 | 4.286 ± 0.041 | 7 |
| 21.87 ± 0.20 | 4.060 ± 0.037 | 3 |
| 23.77 ± 0.20 | 3.740 ± 0.031 | 4 |
| 24.16 ± 0.20 | 3.681 ± 0.030 | 3 |
| 24.31 ± 0.20 | 3.658 ± 0.030 | 3 |
| 24.45 ± 0.20 | 3.638 ± 0.029 | 4 |
| 24.90 ± 0.20 | 3.573 ± 0.028 | 3 |

Substantially pure crystalline sodium cholesteryl sulfate Form H is further disclosed. “Substantially pure,” as described herein, generally refers to a form herein that is present without any appreciable amounts, other than potentially trace levels of other forms of crystalline sodium cholesteryl sulfate. Examples of trace levels include not more than about 10%, 5%, 2%, 1.5%, 1%, 0.5%, 0.25%, 0.1%, or less by weight in total relative to the total amount of crystalline sodium cholesteryl sulfate present.

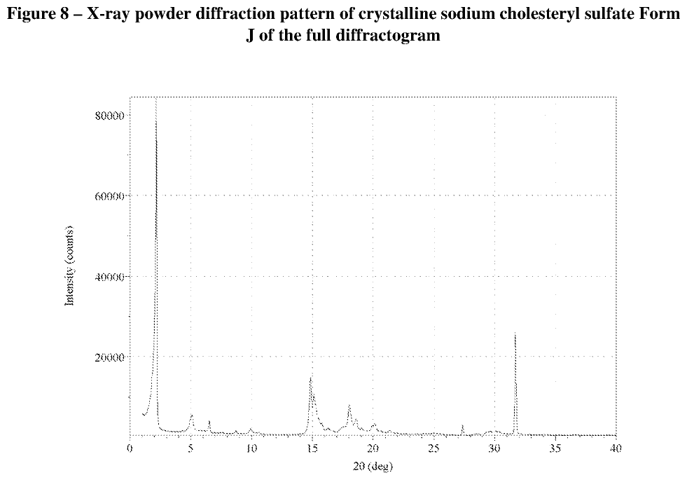

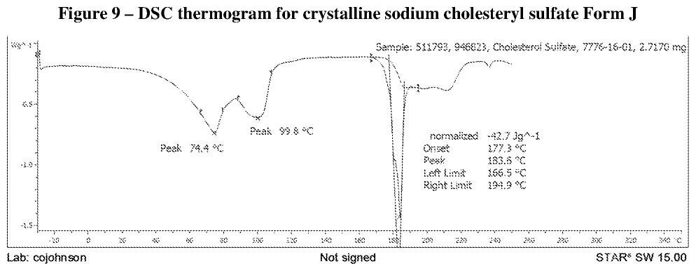

In many embodiments, crystalline sodium cholesteryl sulfate Form J is provided. Without being bound by theory, it is believed that crystalline sodium cholesteryl sulfate Form J is a hydrate. The ¹H-NMR spectrum of crystalline sodium cholesteryl sulfate Form J lacks a noticeable solvent peak and has a 5% weight loss from 40° C. to 121° C. Example 4 sets forth a preparation for crystalline sodium cholesteryl sulfate Form J. An x-ray powder diffraction pattern for crystalline sodium cholesteryl sulfate Form J is set forth in FIG. 8. FIG. 9 is a DSC thermogram for crystalline sodium cholesteryl Form J. Crystalline sodium cholesteryl sulfate Form J may be characterized by various analytical techniques such as x-ray powder diffraction. The x-ray powder diffraction pattern of crystalline sodium cholesteryl sulfate Form J or portions thereof may be used to characterize crystalline sodium cholesteryl sulfate Form J. For example, crystalline sodium cholesteryl sulfate Form J may be characterized by an x-ray powder diffraction pattern having more than one peak between about 1.5°2θ and about 7.0°2θ. In these and other embodiments, the x-ray powder diffraction pattern of crystalline sodium cholesteryl sulfate Form J has not more than three peaks between about 1.5°2θ and about 7.0°2θ. In these and other embodiments, crystalline sodium cholesteryl sulfate Form J has an x-ray powder diffraction pattern comprising a peak between about 5.0°2θ and about 5.2°2θ. In other embodiments, crystalline sodium cholesteryl sulfate Form J may be characterized by an x-ray powder diffraction pattern substantially the same as that found in FIG. 8.

Substantially pure crystalline sodium cholesteryl sulfate Form J is further disclosed. “Substantially pure,” as described herein, generally refers to a form herein that is present without any appreciable amounts, other than potentially trace levels of other forms of crystalline sodium cholesteryl sulfate. Examples of trace levels include not more than about 10%, 5%, 2%, 1.5%, 1%, 0.5%, 0.25%, 0.1%, or less in total relative to the total amount of crystalline sodium cholesteryl sulfate present.

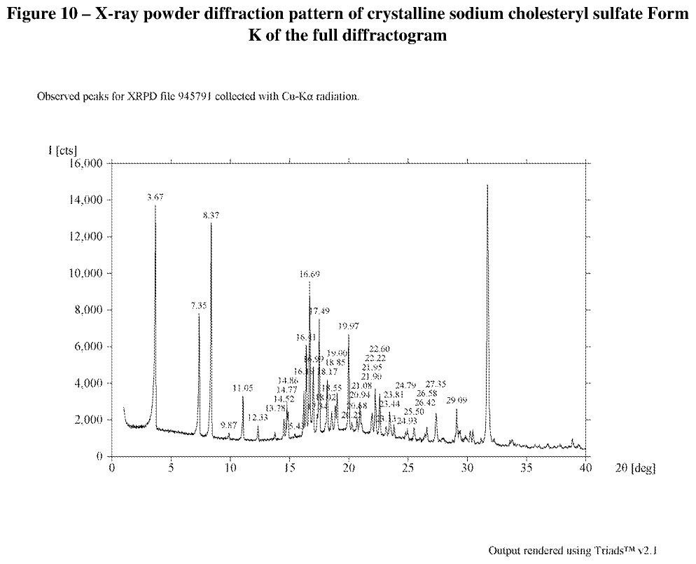

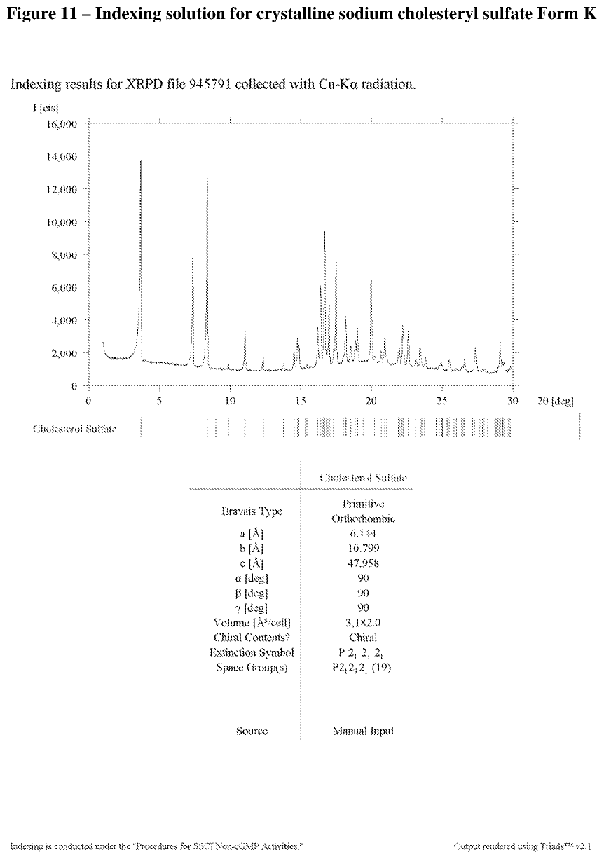

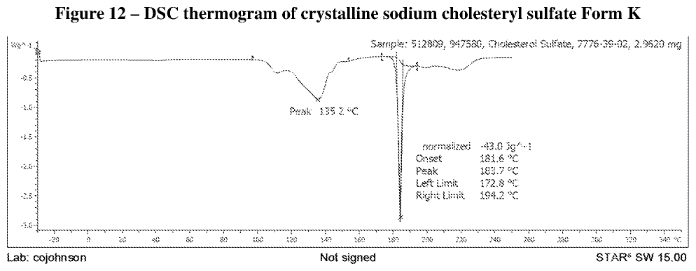

In many embodiments, crystalline sodium cholesteryl sulfate Form K is provided. Without being bound by theory, crystalline sodium cholesteryl sulfate Form K is believed to be a DMF (dimethylformamide) solvate. ¹H-NMR shows 1 mole of DMF present per mole of sodium cholesteryl sulfate. Example 5 sets forth a preparation for crystalline sodium cholesteryl sulfate Form K. An x-ray powder diffraction pattern for crystalline sodium cholesteryl sulfate Form K is set forth in FIG. 10. Table 3 sets forth the observed peaks for FIG. 10. FIG. 11 is the indexing solution for crystalline sodium cholesteryl sulfate Form K, showing the unit cell to be orthorhombic. FIG. 12 is a DSC thermogram of crystalline sodium cholesteryl sulfate Form K.

TABLE 3
Observed peaks for Crystalline Sodium Cholesteryl Sulfate Form K

| 2θ (°) | d-spacing (Å) | Intensity (%) |
|---|---|---|
| 3.67 ± 0.20 | 24.063 ± 1.311 | 100 |
| 7.35 ± 0.20 | 12.012 ± 0.326 | 56 |
| 8.37 ± 0.20 | 10.550 ± 0.251 | 93 |
| 9.87 ± 0.20 | 8.956 ± 0.181 | 9 |
| 11.05 ± 0.20 | 8.000 ± 0.144 | 24 |
| 12.33 ± 0.20 | 7.173 ± 0.116 | 12 |
| 13.78 ± 0.20 | 6.423 ± 0.093 | 9 |
| 14.52 ± 0.20 | 6.096 ± 0.084 | 15 |
| 14.77 ± 0.20 | 5.994 ± 0.081 | 21 |
| 14.86 ± 0.20 | 5.955 ± 0.080 | 18 |
| 15.43 ± 0.20 | 5.739 ± 0.074 | 9 |
| 16.19 ± 0.20 | 5.469 ± 0.067 | 26 |
| 16.41 ± 0.20 | 5.399 ± 0.065 | 44 |
| 16.69 ± 0.20 | 5.308 ± 0.063 | 69 |
| 16.99 ± 0.20 | 5.214 ± 0.061 | 35 |
| 17.34 ± 0.20 | 5.111 ± 0.059 | 16 |
| 17.49 ± 0.20 | 5.066 ± 0.057 | 55 |
| 18.02 ± 0.20 | 4.918 ± 0.054 | 13 |
| 18.17 ± 0.20 | 4.878 ± 0.053 | 30 |
| 18.55 ± 0.20 | 4.780 ± 0.051 | 18 |
| 18.85 ± 0.20 | 4.703 ± 0.049 | 20 |
| 19.00 ± 0.20 | 4.666 ± 0.049 | 25 |
| 19.97 ± 0.20 | 4.442 ± 0.044 | 49 |
| 20.25 ± 0.20 | 4.382 ± 0.043 | 13 |
| 20.68 ± 0.20 | 4.291 ± 0.041 | 16 |
| 20.94 ± 0.20 | 4.240 ± 0.040 | 22 |
| 21.08 ± 0.20 | 4.212 ± 0.040 | 13 |
| 21.90 ± 0.20 | 4.055 ± 0.037 | 16 |
| 21.95 ± 0.20 | 4.046 ± 0.036 | 17 |
| 22.22 ± 0.20 | 3.998 ± 0.036 | 27 |
| 22.60 ± 0.20 | 3.932 ± 0.034 | 25 |
| 23.13 ± 0.20 | 3.842 ± 0.033 | 12 |
| 23.44 ± 0.20 | 3.792 ± 0.032 | 18 |
| 23.81 ± 0.20 | 3.735 ± 0.031 | 13 |
| 24.79 ± 0.20 | 3.589 ± 0.029 | 10 |
| 24.93 ± 0.20 | 3.569 ± 0.028 | 11 |
| 25.50 ± 0.20 | 3.491 ± 0.027 | 11 |
| 26.42 ± 0.20 | 3.371 ± 0.025 | 9 |
| 26.58 ± 0.20 | 3.351 ± 0.025 | 11 |
| 27.35 ± 0.20 | 3.258 ± 0.023 | 17 |
| 29.09 ± 0.20 | 3.067 ± 0.021 | 19 |

Crystalline sodium cholesteryl sulfate Form K may be characterized by various analytical techniques such as x-ray powder diffraction. The x-ray powder diffraction pattern of crystalline sodium cholesteryl sulfate Form K or portions thereof may be used to characterize crystalline sodium cholesteryl sulfate Form K. For example, crystalline sodium cholesteryl sulfate Form K may be characterized by an x-ray powder diffraction pattern comprising a peak at about 3.7°2θ. In these and other embodiments, crystalline sodium cholesteryl sulfate Form K may be characterized by an x-ray powder diffraction pattern comprising a peak at about 3.7°2θ and further comprising one or more peaks at about 7.4°2θ, about 8.4°2θ, about 9.9°2θ, about 11.1°2θ, and about 12.3°2θ.

In other embodiments, crystalline sodium cholesteryl sulfate Form K may be characterized by an x-ray powder diffraction pattern comprising: (i) peaks at about 3.7°2θ, at about 7.4°2θ, and at about 8.4°2θ; or (ii) peaks at about 3.7°2θ, at about 8.4°2θ, and at about 11.1°2θ; or (iii) peaks at about 3.7°2θ, at about 7.4°2θ, at about 8.4°2θ, at about 9.9°2θ, and at about 11.1°θ. In other embodiments, crystalline sodium cholesteryl sulfate Form K may be characterized by an x-ray powder diffraction pattern substantially the same as that found in FIG. 10.

Substantially pure crystalline sodium cholesteryl sulfate Form K is further disclosed. “Substantially pure,” as described herein, generally refers to a form herein that is present without any appreciable amounts, other than potentially trace levels of other forms of crystalline sodium cholesteryl sulfate. Examples of trace levels include not more than about 10%, 5%, 2%, 1.5%, 1%, 0.5%, 0.25%, 0.1%, or less by weight in total relative to the total amount of crystalline sodium cholesteryl sulfate present.

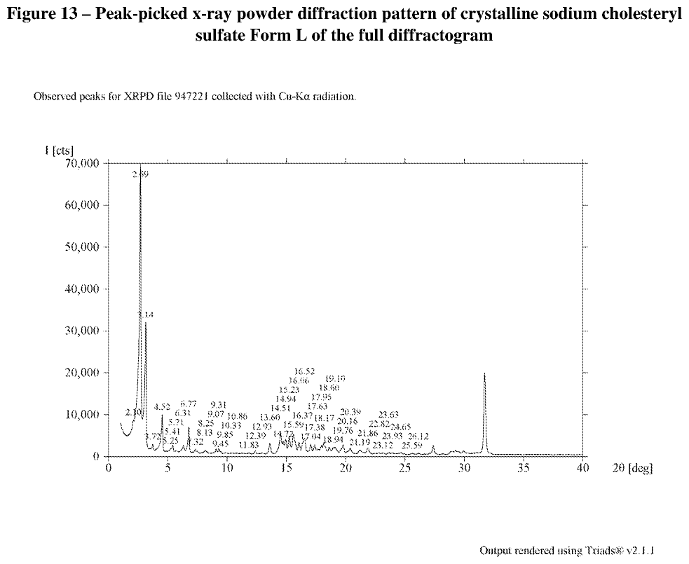

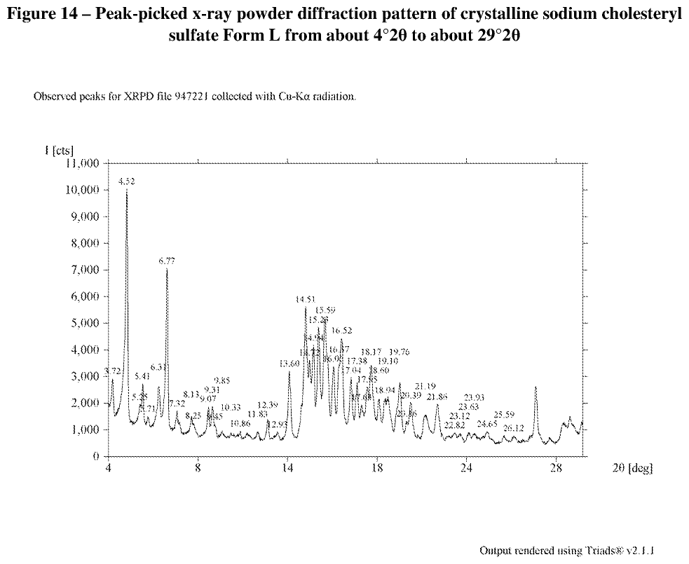

In many embodiments, crystalline sodium cholesteryl sulfate Form L is provided. Without being bound by theory, it is believed that crystalline sodium cholesteryl sulfate Form L is an anhydrate. The ¹H-NMR spectrum showed only negligible amounts of solvent. A preparation of crystalline sodium cholesteryl sulfate Form L is set forth in Example 6. An x-ray powder diffraction pattern for crystalline sodium cholesteryl sulfate Form L is set forth in FIG. 13 which also indicates specifically identified peaks. An expanded set of peaks is set forth in FIG. 14. Table 4 also shows peaks identified in FIG. 13 and FIG. 14.

TABLE 4
Observed peaks for Crystalline Sodium Cholesteryl Sulfate Form L

| º2θ | d space (Å) | Intensity (%) |
|---|---|---|
| 2.10 ± 0.20 | 42.046 ± 4.005 | 13 |
| 2.69 ± 0.20 | 32.847 ± 2.444 | 100 |
| 3.14 ± 0.20 | 28.141 ± 1.794 | 47 |
| 3.72 ± 0.20 | 23.723 ± 1.274 | 4 |
| 4.52 ± 0.20 | 19.550 ± 0.865 | 15 |
| 5.25 ± 0.20 | 16.812 ± 0.640 | 3 |
| 5.41 ± 0.20 | 16.325 ± 0.603 | 4 |
| 5.71 ± 0.20 | 15.477 ± 0.542 | 2 |
| 6.31 ± 0.20 | 13.996 ± 0.443 | 4 |
| 6.77 ± 0.20 | 13.055 ± 0.385 | 10 |
| 7.32 ± 0.20 | 12.074 ± 0.330 | 2 |
| 8.13 ± 0.20 | 10.860 ± 0.267 | 2 |
| 8.25 ± 0.20 | 10.714 ± 0.259 | 2 |
| 9.07 ± 0.20 | 9.738 ± 0.214 | 3 |
| 9.31 ± 0.20 | 9.494 ± 0.204 | 3 |
| 9.45 ± 0.20 | 9.354 ± 0.198 | 2 |
| 9.85 ± 0.20 | 8.976 ± 0.182 | 1 |
| 10.33 ± 0.20 | 8.553 ± 0.165 | 1 |
| 10.86 ± 0.20 | 8.142 ± 0.150 | 1 |
| 11.83 ± 0.20 | 7.473 ± 0.126 | 1 |
| 12.39 ± 0.20 | 7.139 ± 0.115 | 2 |
| 12.93 ± 0.20 | 6.840 ± 0.105 | 1 |
| 13.60 ± 0.20 | 6.506 ± 0.095 | 5 |
| 14.51 ± 0.20 | 6.100 ± 0.084 | 8 |
| 14.72 ± 0.20 | 6.012 ± 0.081 | 5 |
| 14.94 ± 0.20 | 5.925 ± 0.079 | 6 |
| 15.23 ± 0.20 | 5.811 ± 0.076 | 7 |
| 15.59 ± 0.20 | 5.681 ± 0.072 | 8 |
| 16.06 ± 0.20 | 5.514 ± 0.068 | 5 |
| 16.37 ± 0.20 | 5.411 ± 0.066 | 5 |
| 16.52 ± 0.20 | 5.363 ± 0.064 | 6 |
| 17.04 ± 0.20 | 5.199 ± 0.061 | 4 |
| 17.38 ± 0.20 | 5.099 ± 0.058 | 4 |
| 17.63 ± 0.20 | 5.028 ± 0.057 | 3 |
| 17.95 ± 0.20 | 4.937 ± 0.055 | 4 |
| 18.17 ± 0.20 | 4.880 ± 0.053 | 5 |
| 18.60 ± 0.20 | 4.766 ± 0.051 | 3 |
| 18.94 ± 0.20 | 4.682 ± 0.049 | 3 |
| 19.10 ± 0.20 | 4.644 ± 0.048 | 3 |
| 19.76 ± 0.20 | 4.488 ± 0.045 | 4 |
| 20.16 ± 0.20 | 4.402 ± 0.043 | 2 |
| 20.39 ± 0.20 | 4.352 ± 0.042 | 3 |
| 21.19 ± 0.20 | 4.190 ± 0.039 | 2 |
| 21.86 ± 0.20 | 4.062 ± 0.037 | 3 |
| 22.82 ± 0.20 | 3.894 ± 0.034 | 1 |
| 23.12 ± 0.20 | 3.844 ± 0.033 | 1 |
| 23.63 ± 0.20 | 3.762 ± 0.031 | 1 |
| 23.93 ± 0.20 | 3.715 ± 0.031 | 1 |
| 24.65 ± 0.20 | 3.609 ± 0.029 | 1 |
| 25.59 ± 0.20 | 3.478 ± 0.027 | 1 |
| 26.12 ± 0.20 | 3.409 ± 0.026 | 1 |

Crystalline sodium cholesteryl sulfate Form L may be characterized by various analytical techniques such as x-ray powder diffraction. The x-ray powder diffraction pattern of crystalline sodium cholesteryl sulfate Form L or portions thereof may be used to characterize crystalline sodium cholesteryl sulfate Form L. For example, crystalline sodium cholesteryl sulfate Form L may be characterized by an x-ray powder diffraction pattern having three peaks between about 1.9°2θ and about 3.3°2θ; for example, one peak may be at about 2.1°2θ, another at about 2.7°2θ, and another at about 3.1°2θ, and may further comprise one or more peaks at about 4.5°2θ and about 6.8°θ. In various other embodiments, crystalline sodium cholesteryl sulfate Form L may be characterized by an x-ray powder diffraction pattern substantially the same as that found in FIG. 13. In various other embodiments, crystalline sodium cholesteryl sulfate Form L may be characterized by an x-ray powder diffraction pattern substantially the same as that found in FIG. 14, which is a diffraction pattern of an expanded region of FIG. 13.

A DSC thermogram for crystalline sodium cholesteryl sulfate Form L is shown in FIG. 13. It indicates an onset of about 218° C., which may be used to characterize crystalline sodium cholesteryl sulfate Form L alone or in connection with XRPD data used to characterize crystalline sodium cholesteryl sulfate Form L.

Substantially pure crystalline sodium cholesteryl sulfate Form L is further disclosed. “Substantially pure,” as described herein, generally refers to a form herein that is present without any appreciable amounts, other than potentially trace levels of other forms of crystalline sodium cholesteryl sulfate. Examples of trace levels include not more than about 10%, 5%, 2%, 1.5%, 1%, 0.5%, 0.25%, 0.1%, or less by weight in total relative to the total amount of crystalline sodium cholesteryl sulfate present.

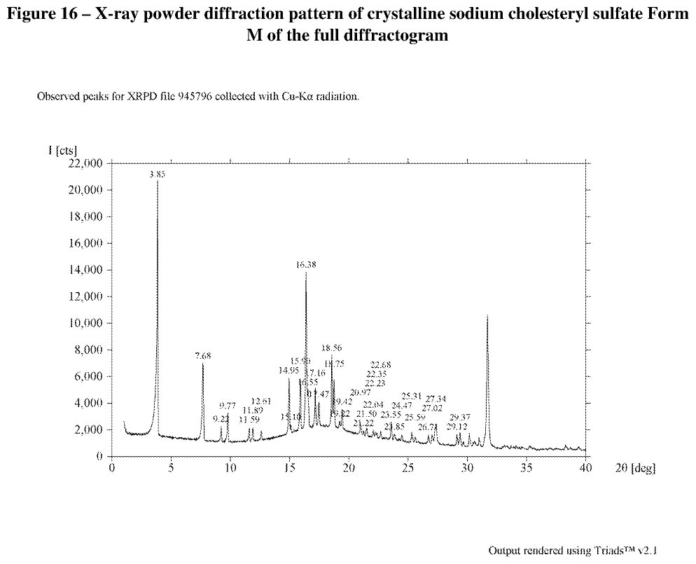

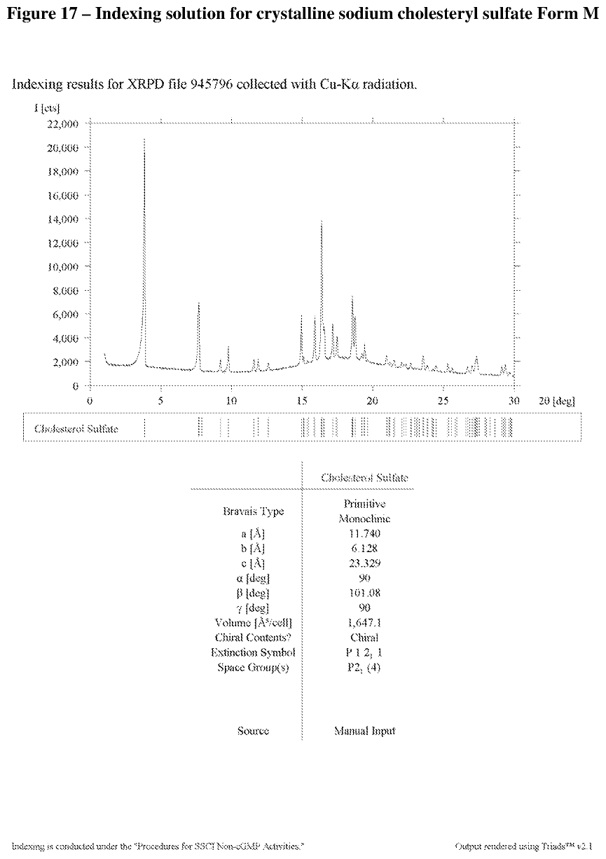

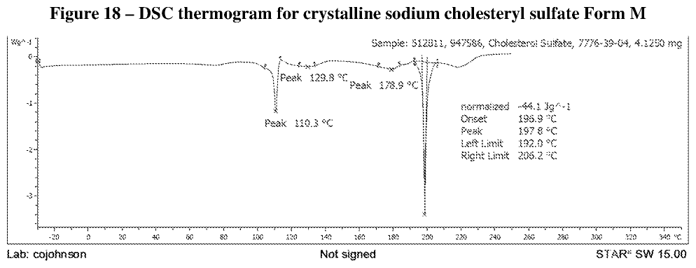

In many embodiments, crystalline sodium cholesteryl sulfate Form M is provided. Without being bound by theory, it is believed that crystalline sodium cholesteryl sulfate Form M is an NMP solvate. ¹H-NMR spectroscopy shows there to be 0.8 moles of NMP per 1 mole of sodium cholesteryl sulfate. An x-ray powder diffraction pattern for crystalline sodium cholesteryl sulfate Form M is set forth in FIG. 16 which also indicates specifically identified peaks. FIG. 17 is the indexing solution for crystalline sodium cholesteryl sulfate Form M indicating a monoclinic unit cell. FIG. 18 shows the DSC thermogram of crystalline sodium cholesteryl sulfate Form M. Example 7 sets forth a preparation of crystalline sodium cholesteryl sulfate Form M. An x-ray powder diffraction pattern for crystalline sodium cholesteryl sulfate Form M is set forth in FIG. 16. Table 5 sets forth specifically identified peaks from FIG. 16.

TABLE 5
Observed peaks for Crystalline Sodium Cholesteryl Sulfate Form M

| 2θ (°) | d-spacing (Å) | Intensity (%) |
|---|---|---|
| 3.85 ± 0.20 | 22.952 ± 1.193 | 100 |
| 7.68 ± 0.20 | 11.496 ± 0.299 | 34 |
| 9.22 ± 0.20 | 9.579 ± 0.207 | 11 |
| 9.77 ± 0.20 | 9.042 ± 0.185 | 16 |
| 11.59 ± 0.20 | 7.632 ± 0.131 | 11 |
| 11.89 ± 0.20 | 7.440 ± 0.125 | 10 |
| 12.61 ± 0.20 | 7.015 ± 0.111 | 9 |
| 14.95 ± 0.20 | 5.921 ± 0.079 | 29 |
| 15.10 ± 0.20 | 5.864 ± 0.077 | 11 |
| 15.90 ± 0.20 | 5.570 ± 0.070 | 28 |
| 16.38 ± 0.20 | 5.407 ± 0.066 | 67 |
| 16.55 ± 0.20 | 5.353 ± 0.064 | 24 |
| 17.16 ± 0.20 | 5.163 ± 0.060 | 25 |
| 17.47 ± 0.20 | 5.073 ± 0.058 | 20 |
| 18.56 ± 0.20 | 4.777 ± 0.051 | 37 |
| 18.75 ± 0.20 | 4.729 ± 0.050 | 28 |
| 19.22 ± 0.20 | 4.614 ± 0.048 | 13 |
| 19.42 ± 0.20 | 4.566 ± 0.047 | 17 |
| 20.97 ± 0.20 | 4.232 ± 0.040 | 12 |
| 21.22 ± 0.20 | 4.184 ± 0.039 | 9 |
| 21.50 ± 0.20 | 4.130 ± 0.038 | 10 |
| 22.04 ± 0.20 | 4.030 ± 0.036 | 10 |
| 22.23 ± 0.20 | 3.996 ± 0.036 | 9 |
| 22.35 ± 0.20 | 3.975 ± 0.035 | 9 |
| 22.68 ± 0.20 | 3.917 ± 0.034 | 9 |
| 23.55 ± 0.20 | 3.775 ± 0.032 | 12 |
| 23.85 ± 0.20 | 3.728 ± 0.031 | 8 |
| 24.47 ± 0.20 | 3.635 ± 0.029 | 8 |
| 25.31 ± 0.20 | 3.516 ± 0.027 | 9 |
| 25.59 ± 0.20 | 3.478 ± 0.027 | 7 |
| 26.71 ± 0.20 | 3.335 ± 0.025 | 8 |
| 27.02 ± 0.20 | 3.297 ± 0.024 | 8 |
| 27.34 ± 0.20 | 3.260 ± 0.023 | 12 |
| 29.12 ± 0.20 | 3.065 ± 0.021 | 8 |
| 29.37 ± 0.20 | 3.039 ± 0.020 | 9 |

Crystalline sodium cholesteryl sulfate Form M may be characterized by various analytical techniques such as x-ray powder diffraction. The x-ray powder diffraction pattern of crystalline sodium cholesteryl sulfate Form M or portions thereof may be used to characterize crystalline sodium cholesteryl sulfate Form M. For example, crystalline sodium cholesteryl sulfate Form M may be characterized by an x-ray powder diffraction pattern comprising a peak at about 3.9°2θ and one or more peaks at about 7.7°2θ, about 9.2°2θ, about 9.8°2θ, about 11.6°2θ, about 11.9°2θ, and about 12.6°θ. In other embodiments, crystalline sodium cholesteryl sulfate Form M may be characterized by an x-ray powder diffraction pattern substantially the same as that found in FIG. 14.

Substantially pure crystalline sodium cholesteryl sulfate Form M is further disclosed. “Substantially pure,” as described herein, generally refers to a form herein that is present without any appreciable amounts, other than potentially trace levels of other forms of crystalline sodium cholesteryl sulfate. Examples of trace levels include not more than about 10%, 5%, 2%, 1.5%, 1%, 0.5%, 0.25%, 0.1%, or less by weight in total relative to the total amount of crystalline sodium cholesteryl sulfate present.

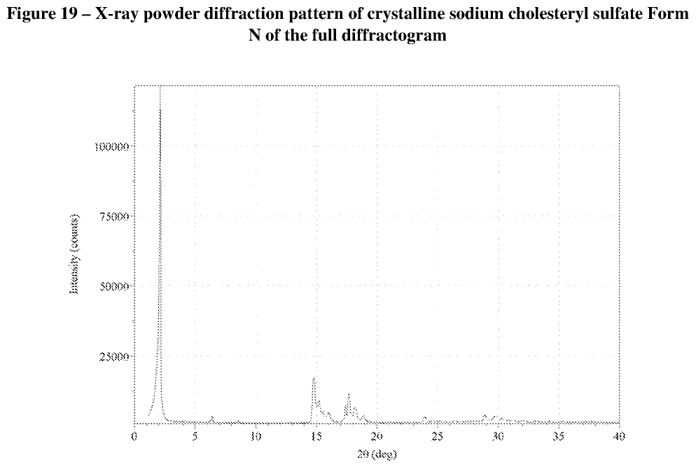

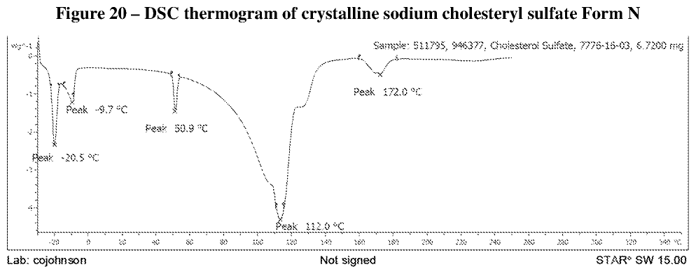

In many embodiments, crystalline sodium cholesteryl sulfate Form N is provided. Without being bound by theory, it is believed crystalline sodium cholesteryl sulfate Form N is a hydrate. ¹H-NMR spectroscopy indicates no visible organic solvent, but water is present in the spectrum. FIG. 20 is a DSC thermogram of crystalline sodium cholesteryl sulfate Form N. Example 8 sets forth a preparation of crystalline sodium cholesteryl sulfate Form N. An x-ray powder diffraction pattern for crystalline sodium cholesteryl sulfate Form N is set forth in FIG. 19.

Crystalline sodium cholesteryl sulfate Form N may be characterized by various analytical techniques such as x-ray powder diffraction. The x-ray powder diffraction pattern of crystalline sodium cholesteryl sulfate Form N or portions thereof may be used to characterize crystalline sodium cholesteryl sulfate Form N. For example, crystalline sodium cholesteryl sulfate Form N may be characterized by an x-ray powder diffraction pattern having a single peak between about 1.5°2θ and about 6.0°θ. In these and other embodiments, crystalline sodium cholesteryl sulfate Form N may be characterized by a peak between about 2.0°2θ and about 2.2°θ. In these and other embodiments, crystalline sodium cholesteryl sulfate Form N does not have a peak at about 4.0°θ. In other embodiments, crystalline sodium cholesteryl sulfate Form N may be characterized by an x-ray powder diffraction pattern substantially the same as that found in FIG. 19.

Substantially pure crystalline sodium cholesteryl sulfate Form N is further disclosed. “Substantially pure,” as described herein, generally refers to a form herein that is present without any appreciable amounts, other than potentially trace levels of other forms of crystalline sodium cholesteryl sulfate. Examples of trace levels include not more than about 10%, 5%, 2%, 1.5%, 1%, 0.5%, 0.25%, 0.1%, or less by weight in total relative to the total amount of crystalline sodium cholesteryl sulfate present.

In many embodiments, crystalline sodium cholesteryl sulfate Form B is provided. Without being bound by theory, it is believed crystalline sodium cholesteryl sulfate Form B is a solvate with methanol. Example 2 sets forth a preparation of crystalline sodium cholesteryl sulfate Form B. An x-ray powder diffraction pattern for crystalline sodium cholesteryl sulfate Form B is set forth in FIG. 22.

Crystal sodium cholesteryl sulfate Form B may be characterized by various analytical techniques such as x-ray powder diffraction. The x-ray powder diffraction pattern of crystalline sodium cholesteryl sulfate Form B or portions thereof may be used to characterize crystalline sodium cholesteryl sulfate Form B. For example, crystalline sodium cholesteryl sulfate Form B may be characterized by an x-ray powder diffraction pattern comprising one or more peaks between about 2.0°2θ and about 2.5°2θ, such as a single peak between about 2.0°2θ and about 2.5°θ. In embodiments with a single peak, the peak may be between about 2.1°2θ and about 2.4°2θ and in some embodiments between about 2.2°2θ and about 2.3°θ. In these and other embodiments, the x-ray powder diffraction pattern of crystalline sodium cholesteryl sulfate Form B lacks any x-ray powder diffraction peak between about 3.8°2θ and 4.2°θ. In other embodiments, crystalline sodium cholesteryl sulfate Form B may be characterized by an x-ray powder diffraction pattern substantially the same as that found in FIG. 22.

Substantially pure crystalline sodium cholesteryl sulfate Form B is further disclosed. “Substantially pure,” as described herein, generally refers to a form herein that is present without any appreciable amounts, other than potentially trace levels of other forms of crystalline sodium cholesteryl sulfate. Examples of trace levels include not more than about 10%, 5%, 2%, 1.5%, 1%, 0.5%, 0.25%, 0.1%, or less by weight in total relative to the total amount of crystalline sodium cholesteryl sulfate present.

This disclosure also relates to pharmaceutical compositions containing crystalline sodium cholesteryl sulfate as disclosed herein. Such pharmaceutical compositions are comprised of one or more pharmaceutically acceptable excipients and crystalline sodium cholesteryl sulfate as set forth in the present disclosure. Such pharmaceutical compositions may be administered orally or configured to be delivered as any effective conventional dosage unit forms, including, for example, immediate, slow and timed-release oral preparations, parenterally, topically, nasally, ophthalmically, optically, sublingually, rectally, vaginally, and the like.

The present disclosure further includes mixtures of forms of crystalline sodium cholesteryl sulfate. For examples, mixtures of two or more of crystalline sodium cholesteryl sulfate Form B, crystalline sodium cholesteryl sulfate Form F, crystalline sodium cholesteryl sulfate Form H, crystalline sodium cholesteryl sulfate Form J, crystalline sodium cholesteryl sulfate Form K, crystalline sodium cholesteryl sulfate Form L, crystalline sodium cholesteryl sulfate Form M, and crystalline sodium cholesteryl sulfate Form N are provided. The amount of each form present in such mixtures ranges from, for example, about 0.1% to about 99.9% by weight. Other ranges include about 0.1% to about 95%, about 0.1% to about 90%, about 0.1% to about 85%, about 0.1% to about 80%, about 0.1% to about 75%, about 0.1% to about 70%, about 0.1% to about 65%, about 0.1% to about 60%, about 0.1% to about 55%, about 0.1% to about 50%, about 0.1% to about 45%, about 0.1% to about 40%, about 0.1% to about 35%, about 0.1% to about 30%, about 0.1% to about 25%, about 0.1% to about 20%, about 0.1% to about 15%, and about 0.1% to about 10% by weight. Other ranges include about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, and about 0.1% to about 1% by weight. Additional ranges include about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, and about 0.1% to about 0.2% by weight. Such mixtures may also be present in pharmaceutical compositions for the comprising one or more pharmaceutically acceptable excipients.

The present disclosure further includes methods and uses for treating diseases in humans such as one or more of hypercholesterolemia, hypertriglyceridemia, and conditions related to fat-accumulation and inflammation (e.g., non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, and atherosclerosis) with effective amounts crystalline sodium cholesteryl sulfate and/or pharmaceutical compositions comprising crystalline sodium cholesteryl sulfate of the present disclosure. Such crystalline sodium cholesteryl sulfate solid forms include one or more of crystalline sodium cholesteryl sulfate Form B, crystalline sodium cholesteryl sulfate Form F, crystalline sodium cholesteryl sulfate Form H, crystalline sodium cholesteryl sulfate Form J, crystalline sodium cholesteryl sulfate Form K, crystalline sodium cholesteryl sulfate Form L, crystalline sodium cholesteryl sulfate Form M, and crystalline sodium cholesteryl sulfate Form N are provided.

The present disclosure may be further described by one or more of the non-limiting clauses that follow.

Clause 1. Crystalline sodium cholesteryl sulfate.

Clause 2. A hydrate of crystalline sodium cholesteryl sulfate.

Clause 3. A solvate of crystalline sodium cholesteryl sulfate.

Clause 4. An anhydrate of crystalline sodium cholesteryl sulfate.

Clause 5. The solvate of clause 3, wherein the solvate is an alcohol-solvate.

Clause 6. The solvate of clause 4, wherein an alcohol is methanol.

Clause 7. Crystalline sodium cholesteryl sulfate Form F.

Clause 8. Crystalline sodium cholesteryl sulfate of any one of clauses 1, 4, or 7, having an x-ray powder diffraction pattern with two peaks between about 1.9°2θ and about 3.3°2θ.

Clause 9. The crystalline sodium cholesteryl sulfate of clause 8, having an x-ray powder diffraction pattern wherein one of the x-ray powder diffraction peaks is at about 2.7°2θ.

Clause 10. The crystalline sodium cholesteryl sulfate of any one of clauses 8-9, having an x-ray powder diffraction pattern wherein one of the x-ray powder diffraction peaks is at about 3.0°2θ.

Clause 11. The crystalline sodium cholesteryl sulfate of any one of clauses 8-10, having an x-ray powder diffraction pattern further comprising one or more peaks at about 6.0°2θ and about 6.4°2θ.

Clause 12. The crystalline sodium cholesteryl sulfate Form F of any one of clauses 8-11, having an x-ray powder diffraction pattern wherein one of the x-ray powder diffraction peaks is at about 6.0°2θ.

Clause 13. The crystalline sodium cholesteryl sulfate Form F of any one of clauses 8-12, having an x-ray powder diffraction pattern wherein one of the x-ray powder diffraction peaks is at about 6.4°2θ.

Clause 14. Crystalline sodium cholesteryl sulfate of any one of clauses 1, 4, or 7-13, having a DSC thermogram with a peak onset of about 217° C.

Clause 15. Crystalline sodium cholesteryl sulfate of any one of clauses 1, 4, or 7-14 having an x-ray powder diffraction pattern substantially the same as that of FIG. 1.

Clause 16. Crystalline sodium cholesteryl sulfate of any one of clauses 1, 4, or 7-15 having a portion of an x-ray powder diffraction pattern substantially the same as that of FIG. 2.

Clause 17. Crystalline sodium cholesteryl sulfate of any one of clauses 1, 4, or 7-16 having a DSC thermogram substantially the same as that of FIG. 3.

Clause 18. Crystalline sodium cholesteryl sulfate Form L.

Clause 19. Crystalline sodium cholesteryl sulfate of any one of clauses 1, 4, or 18 having an x-ray powder diffraction pattern comprising three peaks between about 1.9°2θ and about 3.3°2θ.

Clause 20. The crystalline sodium cholesteryl sulfate of clause 19, having an x-ray powder diffraction pattern wherein one of the x-ray powder diffraction peaks is at about 2.1°2θ.

Clause 21. The crystalline sodium cholesteryl sulfate of any one of clauses 19-20, having an x-ray powder diffraction pattern wherein one of the x-ray powder diffraction peaks is at about 2.7°2θ.

Clause 22. The crystalline sodium cholesteryl sulfate of any one of clauses 19-21, having an x-ray powder diffraction pattern wherein one of the x-ray powder diffraction peaks is at about 3.1°2θ.

Clause 23. The crystalline sodium cholesteryl sulfate of any one of clauses 19-22, having an x-ray powder diffraction pattern further comprising one or more peaks at about 4.5°2θ and about 6.8°2θ.

Clause 24. The crystalline sodium cholesteryl sulfate Form L of clauses 23, having an x-ray powder diffraction pattern wherein a peak is at about 4.5°2θ.

Clause 25. The crystalline sodium cholesteryl sulfate Form L of clauses 23-24, having an x-ray powder diffraction pattern wherein a peak is at about 6.8°2θ.

Clause 26. Crystalline sodium cholesteryl sulfate of any one of clauses 1, 4, or 19-25, having a DSC thermogram with a peak onset of about 218° C.

Clause 27. Crystalline sodium cholesteryl sulfate of any one of clauses 1, 4, or 19-26 having an x-ray powder diffraction pattern substantially the same as that of FIG. 13.

Clause 28. Crystalline sodium cholesteryl sulfate of any one of clauses 1, 4, or 19-27, having a portion of an x-ray powder diffraction pattern substantially the same as that of FIG. 14.

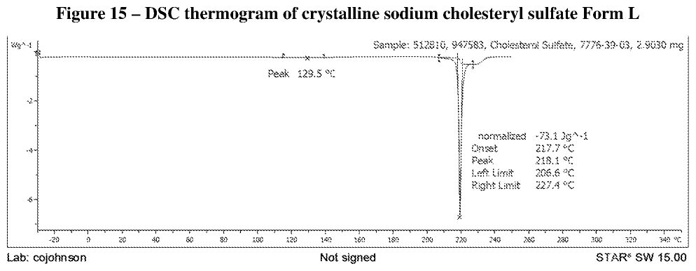

Clause 29. Crystalline sodium cholesteryl sulfate of any one of clauses 1, 4, or 19-28, having a DSC thermogram substantially the same as that of FIG. 15.

Clause 30. Crystalline sodium cholesteryl sulfate Form J.

Clause 31. Crystalline sodium cholesteryl sulfate of any one of clauses 1, 2, or 30, having an x-ray powder diffraction pattern with more than one peak between about 1.5°2θ and about 7.0°2θ.

Clause 32. Crystalline sodium cholesteryl sulfate of clause 31, having an x-ray powder diffraction pattern with not more than three peaks between about 1.5°2θ and about 7.0°2θ.

Clause 33. Crystalline sodium cholesteryl sulfate of any one of clauses 1, 2, or 30-32, having an x-ray powder diffraction pattern substantially the same as that of FIG. 8.

Clause 34. Crystalline sodium cholesteryl sulfate Form N.

Clause 35. Crystalline sodium cholesteryl sulfate of any one of clauses 1, 2, or 34 having, an x-ray powder diffraction pattern with a single peak between about 1.5°2θ and about 6.0°2θ.

Clause 36. Crystalline sodium cholesteryl sulfate of any one of clauses 1, 2, or 34-35 having an x-ray powder diffraction pattern substantially the same as that of FIG. 19.

Clause 37. Crystalline sodium cholesteryl sulfate Form H.

Clause 38. Crystalline sodium cholesteryl sulfate of any one of clauses 1, 4, 5, or 37, having an x-ray powder diffraction pattern comprising a peak at about 2.2°2θ.

Clause 39. Crystalline sodium cholesteryl sulfate of any one of clauses 1, 4, 5, or 37-38, having an x-ray powder diffraction pattern comprising a peak at about 3.9°2θ.

Clause 40. Crystalline sodium cholesteryl sulfate of any one of clauses 1, 4, 5, or 37-39, having an x-ray powder diffraction pattern comprising a peak at about 4.0°2θ.

Clause 41. Crystalline sodium cholesteryl sulfate of any one of clauses 38-40, having an x-ray powder diffraction pattern further comprising one or more peaks at about 4.8°2θ, at about 5.2°2θ, and at about 6.5°2θ.

Clause 42. Crystalline sodium cholesteryl sulfate Form H having an x-ray powder diffraction pattern comprising a peak at about 2.2°2θ and a peak at about 4.8°2θ.

Clause 43. Crystalline sodium cholesteryl sulfate Form H having an x-ray powder diffraction pattern comprising a peak at about 2.2°2θ and a peak at about 5.2°2θ.

Clause 44. Crystalline sodium cholesteryl sulfate Form H having an x-ray powder diffraction pattern comprising a peak at about 2.2°2θ and a peak at about 6.5°2θ.

Clause 45. The solvate of clause 3, wherein the solvate is a DMF or NMP solvate.

Clause 46. The solvate of clause 45, wherein the solvate is a DMF solvate.

Clause 47. Crystalline sodium cholesteryl sulfate Form K.

Clause 48. Crystalline sodium cholesteryl sulfate of any one of clauses 1, 3, or 47, having an x-ray powder diffraction pattern comprising a peak at about 3.7°2θ.

Clause 49. Crystalline sodium cholesteryl sulfate of clause 48, having an x-ray powder diffraction pattern further comprising one or more peaks at about 7.4°2θ, at about 8.4°2θ, at about 9.9°2θ, at about 11.1°2θ, and at about 12.3°2θ.

Clause 50. Crystalline sodium cholesteryl sulfate Form K of clause 48, having an x-ray powder diffraction pattern further comprising a peak at about 7.4°2θ.

Clause 51. Crystalline sodium cholesteryl sulfate Form K of clause 48, having an x-ray powder diffraction pattern further comprising a peak at about 8.4°2θ.

Clause 52. Crystalline sodium cholesteryl sulfate Form K of clause 48, having an x-ray powder diffraction pattern further comprising a peak at about 9.9°2θ.

Clause 53. Crystalline sodium cholesteryl sulfate Form K of clause 48, having an x-ray powder diffraction pattern further comprising a peak at about 11.1°2θ.

Clause 54. Crystalline sodium cholesteryl sulfate Form K of clause 48, having an x-ray powder diffraction pattern further comprising a peak at about 12.3°2θ.

Clause 55. Crystalline sodium cholesteryl sulfate Form K of clause 48, having an x-ray powder diffraction pattern further comprising a peak at about 7.4°2θ and a peak at about 8.4°2θ.

Clause 56. Crystalline sodium cholesteryl sulfate Form K of clause 48, having an x-ray powder diffraction pattern further comprising a peak at about 8.4°2θ and a peak at about 11.1°2θ.

Clause 57. Crystalline sodium cholesteryl sulfate Form K of clause 48, having an x-ray powder diffraction pattern further comprising a peak at about 7.4°2θ, a peak at about 8.4°2θ, a peak at about 9.9°2θ, and a peak at about 11.1°2θ.

Clause 58. Crystalline sodium cholesteryl sulfate of any one of clauses 1, 3, or 47, having an x-ray powder diffraction pattern substantially the same as that of FIG. 10.

Clause 59. The solvate of clause 45, wherein the solvate is an NMP solvate.

Clause 60. Crystalline sodium cholesteryl sulfate Form M.

Clause 61. Crystalline sodium cholesteryl sulfate of any one of clauses 1, 3, or 60, having an x-ray powder diffraction pattern comprising a peak at about 3.9°2θ.

Clause 62. The crystalline sodium cholesteryl sulfate of clause 61, having an x-ray powder diffraction pattern further comprising one or more peaks at about 7.7°2θ, at about 9.2°2θ, at about 9.8°2θ, at about 11.6°2θ, at about 11.9°2θ, and at about 12.6°2θ.

Clause 63. Crystalline sodium cholesteryl sulfate Form M of clause 60, having an x-ray powder diffraction pattern comprising a peak at about 3.9°2θ and at about 7.7°2θ.

Clause 64. Crystalline sodium cholesteryl sulfate Form M of clause 60, having an x-ray powder diffraction pattern comprising a peak at about 3.9°2θ and at about 9.2°2θ.

Clause 65. Crystalline sodium cholesteryl sulfate Form M of clause 60, having an x-ray powder diffraction pattern comprising a peak at about 3.9°2θ and at about 9.8°2θ.

Clause 66. Crystalline sodium cholesteryl sulfate Form M of clause 60, having an x-ray powder diffraction pattern comprising a peak at about 3.9°2θ and at about 11.6°2θ.

Clause 67. Crystalline sodium cholesteryl sulfate Form M of clause 60, having an x-ray powder diffraction pattern comprising a peak at about 3.9°2θ and at about 11.9°2θ.

Clause 68. Crystalline sodium cholesteryl sulfate Form M of clause 60, having an x-ray powder diffraction pattern comprising a peak at about 3.9°2θ and at about 12.6°2θ.

Clause 69. Crystalline sodium cholesteryl sulfate Form M of clause 61, having an x-ray powder diffraction pattern further comprising a peak at about 7.7°2θ and a peak at about 9.8°2θ.

Clause 70. The crystalline sodium cholesteryl sulfate of any one of clauses 1, 3, or 60 having an x-ray powder diffraction pattern substantially the same as that of FIG. 16.

Clause 71. The solvate of clause 3, wherein the molar ratio of solvent to sodium cholesteryl sulfate is 1:1.

Clause 72. Crystalline sodium cholesteryl sulfate Form B.

Clause 73. The crystalline sodium cholesteryl sulfate of clause 72, having an x-ray powder diffraction pattern comprising one or more peaks between about 2.0°2θ and about 2.5°2θ.

Clause 74. The crystalline sodium cholesteryl sulfate of clause 72, having an x-ray powder diffraction pattern wherein there is a single peak between about 2.0°2θ and about 2.5°2θ.

Clause 75. The crystalline sodium cholesteryl sulfate of clause 74, having an x-ray powder diffraction pattern wherein the single peak is between about 2.1°2θ and about 2.4°2θ.

Clause 76. The crystalline sodium cholesteryl sulfate of clause 74, having an x-ray powder diffraction pattern wherein the single peak is between about 2.2°2θ and about 2.3°2θ.

Clause 77. The crystalline sodium cholesteryl sulfate of any one of clauses 72-76, having an x-ray powder diffraction pattern wherein there is peak between about 3.8°2θ and 4.2°2θ.

Clause 78. The crystalline sodium cholesteryl sulfate of any one of clauses 1, 3, 5, 6 or 72 having an x-ray powder diffraction pattern substantially the same as that found in FIG. 22.

Clause 79. A process for making crystalline sodium cholesteryl sulfate Form F comprising the step of slurrying solid sodium cholesteryl sulfate in ethanol for sufficient time to crystallize crystalline sodium cholesteryl sulfate Form F.

Clause 80. The process of clause 80 wherein the crystalline sodium cholesteryl sulfate Form F is isolated.

Clause 81. The process of any one of clause 80 or clause 81, wherein the crystalline sodium cholesteryl sulfate Form F is dried.

Clause 82. A process for making crystalline sodium cholesteryl sulfate Form H comprising the steps of combining solid sodium cholesteryl sulfate in NMP to form a solution, sonicating the solution, and adding water to the solution for sufficient time to crystallize crystalline sodium cholesteryl sulfate Form H.

Clause 83. The process of clause 83 wherein the crystalline sodium cholesteryl sulfate Form H is isolated.

Clause 84. The process of clause 83 or clause 84, wherein the crystalline sodium cholesteryl sulfate Form H is dried.

Clause 85. Crystalline sodium cholesteryl sulfate Form H made by the processes of any one of clauses 83-85.

Clause 86. A process for making crystalline sodium cholesteryl sulfate Form K comprising the step of slurrying solid sodium cholesteryl sulfate in DMF for sufficient time to crystallize crystalline sodium cholesteryl sulfate Form K.

Clause 87. The process of clause 87, wherein the crystalline sodium cholesteryl sulfate Form K is isolated.

Clause 88. The process of clause 87 or clause 88, wherein the crystalline sodium cholesteryl sulfate Form K is dried.

Clause 89. Crystalline sodium cholesteryl sulfate Form K made by the processes of any one of clauses 87-89.

Clause 90. A process for making crystalline sodium cholesteryl sulfate Form L comprising the step of slurrying solid sodium cholesteryl sulfate in ethanol for sufficient time to crystallize crystalline sodium cholesteryl sulfate Form L.

Clause 91. The process of clause 91, wherein the crystalline sodium cholesteryl sulfate Form L is isolated.

Clause 92. The process of clause 91 or clause 92, wherein the crystalline sodium cholesteryl sulfate Form L is dried.

Clause 93. Crystalline sodium cholesteryl sulfate Form L made by the processes of any one of clauses 91-93.

Clause 94. A process for making crystalline sodium cholesteryl sulfate Form M comprising the step of slurrying solid sodium cholesteryl sulfate in NMP for sufficient time to crystallize crystalline sodium cholesteryl sulfate Form M.

Clause 95. The process of clause 95, wherein the crystalline sodium cholesteryl sulfate Form M is isolated.

Clause 96. The process of clause 94 or clause 95, wherein the crystalline sodium cholesteryl sulfate Form M is dried.

Clause 97. The process of any one of clauses 95-97, wherein the solid sodium crystalline sulfate is crystalline sodium cholesteryl sulfate Form M.

Clause 98. Crystalline sodium cholesteryl sulfate Form M made by the processes of any one of clauses 95-98.

Clause 99. A mixture of two or more of crystalline sodium cholesteryl sulfate of any one of clauses 1-78, 85, 89, 93, 98, 107-108, 111-113, 115-119 or 122-123.

Clause 100. A mixture of two or more of crystalline sodium cholesteryl sulfate Form B, crystalline sodium cholesteryl sulfate Form F, crystalline sodium cholesteryl sulfate Form L, crystalline sodium cholesteryl sulfate Form J, crystalline sodium cholesteryl sulfate Form N, crystalline sodium cholesteryl sulfate Form H, crystalline sodium cholesteryl sulfate Form K, and crystalline sodium cholesteryl sulfate Form M.

Clause 101. A pharmaceutical composition comprising crystalline sodium cholesteryl sulfate.

Clause 102. A pharmaceutical composition comprising two or more solid forms of crystalline sodium cholesteryl sulfate.

Clause 103. A pharmaceutical composition comprising one or more of crystalline sodium cholesteryl sulfate Form B, crystalline sodium cholesteryl sulfate Form F, crystalline sodium cholesteryl sulfate Form L, crystalline sodium cholesteryl sulfate Form J, crystalline sodium cholesteryl sulfate Form N, crystalline sodium cholesteryl sulfate Form H, crystalline sodium cholesteryl sulfate Form K, and crystalline sodium cholesteryl sulfate Form M and one or more pharmaceutically acceptable excipients.

Clause 104. A method of treating or preventing one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis, comprising administering to a patient in need thereof an effective amount of a compound or pharmaceutical composition thereof comprising crystalline sodium cholesteryl sulfate.

Clause 105. A method of treating or preventing one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis, comprising administering to a patient in need thereof an effective amount of a compound or pharmaceutical composition thereof comprising crystalline sodium cholesteryl sulfate of any one of clauses 1-78, 85, 89, 93, 98, 107-108, 111-113, 115-119 or 122-123.

Clause 106. A method of treating one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis, comprising administering to a patient in need thereof an effective amount of a compound or pharmaceutical composition thereof of one or more of crystalline sodium cholesteryl sulfate Form B, crystalline sodium cholesteryl sulfate Form F, crystalline sodium cholesteryl sulfate Form L, crystalline sodium cholesteryl sulfate Form J, crystalline sodium cholesteryl sulfate Form N, crystalline sodium cholesteryl sulfate Form H, crystalline sodium cholesteryl sulfate Form K, and crystalline sodium cholesteryl sulfate Form M.

Clause 107. Stable crystalline sodium cholesteryl sulfate.

Clause 108. Stable crystalline sodium cholesteryl sulfate of any one of clauses 1-78, 85, 89, 93, 98, 107-108, 111-113, 115-119 or 122-123.

Clause 109. The use of one or more crystalline sodium cholesteryl sulfate solid forms and optionally one or more pharmaceutically acceptable excipients, for treating host mammal with hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis.

Clause 110. The use of one or more of crystalline sodium cholesteryl sulfate Form B, crystalline sodium cholesteryl sulfate Form F, crystalline sodium cholesteryl sulfate Form H, crystalline sodium cholesteryl sulfate Form J, crystalline sodium cholesteryl sulfate Form K, crystalline sodium cholesteryl sulfate Form L, crystalline sodium cholesteryl sulfate Form M, or crystalline sodium cholesteryl sulfate Form N and optionally one or more pharmaceutically acceptable excipients, for treating host mammal with hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis.

Clause 111. The crystalline sodium cholesteryl sulfate of any one of clauses 1 or 37 having an x-ray powder diffraction pattern substantially the same as that of FIG. 5.

Clause 112. The crystalline sodium cholesteryl sulfate of any one of clauses 1 or 37 having an x-ray powder diffraction pattern substantially the same as that of FIG. 6.

Clause 113. The crystalline sodium cholesteryl sulfate of any one of clauses 1 or 34 having an x-ray powder diffraction pattern substantially the same as that of FIG. 19.

Clause 114. A process for making crystalline sodium cholesteryl sulfate Form B.

Clause 115. The crystalline sodium cholesteryl sulfate of any one of clauses 34-35 having an x-ray powder diffraction patter comprising a peak between about 2.0°2θ and about 2.2°2θ.

Clause 116. The crystalline sodium cholesteryl sulfate of any one of clauses 34-36 and 122 having an x-ray powder diffraction pattern with no peak at about 4.0°2θ.

Clause 117. The crystalline sodium cholesteryl sulfate of any one of clauses 30-32, having an x-ray powder diffraction pattern comprising a peak between about 5.0°2θ and about 5.2°2θ.

Clause 118. Substantially pure crystalline sodium cholesteryl sulfate.

Clause 119. Substantially pure crystalline sodium cholesteryl sulfate selected from crystalline sodium cholesteryl sulfate Form B, crystalline sodium cholesteryl sulfate Form F, crystalline sodium cholesteryl sulfate Form H, crystalline sodium cholesteryl sulfate Form J, crystalline sodium cholesteryl sulfate Form K, crystalline sodium cholesteryl sulfate Form L, crystalline sodium cholesteryl sulfate Form M, and crystalline sodium cholesteryl sulfate Form N.

Clause 120. A process for making crystalline sodium cholesteryl sulfate Form N comprising the step of exposing crystalline sodium cholesteryl sulfate to water vapor for sufficient time to crystallize crystalline sodium cholesteryl sulfate Form N.

Clause 121. A process for making crystalline sodium cholesteryl sulfate Form J comprising the step of exposing crystalline sodium cholesteryl sulfate to acetonitrile vapor for sufficient time to crystallize crystalline sodium cholesteryl sulfate Form J.

Clause 122. Crystalline sodium cholesteryl sulfate Form N made by the process of clause 120.

Clause 123. Crystalline sodium cholesteryl sulfate Form J made by the process of clause 121.

Clause 124. Crystalline sodium cholesteryl sulfate as defined in any one of clauses 1-78, 85, 89, 93, 98, 107-108, 111-113, 115-119, 122-123, a mixture of crystalline sodium cholesteryl sulfate as defined in any one clauses 99-100, or a pharmaceutical composition of crystalline sodium cholesteryl sulfate as defined in any one of clauses 101-103, for us as a medicament.

Clause 125. Crystalline sodium cholesteryl sulfate as defined in any one of clauses 1-78, 85, 89, 93, 98, 107-108, 111-113, 115-119, 122-123, a mixture of crystalline sodium cholesteryl sulfate as defined in any one of clauses 99-100, or a pharmaceutical composition of crystalline sodium cholesteryl sulfate as defined in any one of clauses 101-103, for use in a method of treating or preventing one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis.

### EXAMPLES

### Example 1—Preparation of Crystalline Sodium Cholesteryl Sulfate Form F

A slurry of 87.1 mg crystalline sodium cholesteryl sulfate purchased from Avanti Polar Lipids, Inc. in 1 mL of ethanol was magnetically stirred at 55° C. After 1 day, the slurry was too thick to stir and an additional 2 mL of EtOH was added. The slurry continued to stir for 6 days at 55° C. Crystalline sodium cholesteryl sulfate Form F was harvested by decanting the excess liquor after centrifugation and drying under vacuum at ambient temperature for approximately 2 days. FIGS. 1 and 2 are x-ray powder diffraction patterns of crystalline sodium cholesteryl sulfate Form F from this Example 1.

### Example 2—Preparation of Crystalline Sodium Cholesteryl Sulfate Form B

A slurry of crystalline sodium cholesteryl sulfate purchased from Avanti Polar Lipids, Inc. (68.2 mg) in 1 mL of MeOH was magnetically stirred at ambient conditions for 14 days. The mixture was centrifuged and the clear supernatant decanted off. The damp sediment was crystalline sodium cholesteryl sulfate Form B.

### Example 3—Preparation of Crystalline Sodium Cholesteryl Sulfate Form H

A hazy solution was generated with 112.4 mg crystalline sodium cholesteryl sulfate purchased from Avanti Polar Lipids, Inc. in 8 mL of NMP with sonication. A 2.5-mL aliquot was filtered with a 0.2-μm nylon filter to provide a clear solution. Two 500-μL aliquots of water were added to the filtered solution with gentle stirring until hazy and then magnetically stirred at ambient temperature for 1 day. Crystalline sodium cholesteryl sulfate Form H was harvested from the resulting slurry by positive pressure filtration onto a 0.2-μm nylon filter and flushed with 20-mL volumes of air five times. FIGS. 5 and 6 are x-ray powder diffraction patterns of crystalline sodium cholesteryl sulfate Form H from this Example 3.

### Example 4—Preparation of Crystalline Sodium Cholesteryl Sulfate Form J

Crystalline sodium cholesteryl sulfate purchased from Avanti Polar Lipids, Inc. (47.9 mg) was exposed to acetonitrile (ACN) vapor within a closed vessel for 7 days at ambient temperature to provide crystalline sodium cholesteryl sulfate Form J.

### Example 5—Preparation of Crystalline Sodium Cholesteryl Sulfate Form K

A slurry of 93.4 mg crystalline sodium cholesteryl sulfate purchased from Avanti Polar Lipids, Inc. in 1 mL of dimethylformamide (DMF) was magnetically stirred at ambient temperature. After 1 day, the slurry was too thick to stir and an additional 1 mL of DMF was added. The slurry continued to stir for 13 days at ambient temperature. Crystalline sodium cholesteryl sulfate Form K was harvested damp by decanting the excess liquor after centrifugation. FIG. 10 is an x-ray powder diffraction pattern of crystalline sodium cholesteryl sulfate Form K from this Example 5, and FIG. 11 is the indexing solution from that x-ray powder diffraction pattern.

### Example 6—Preparation of Crystalline Sodium Cholesteryl Sulfate Form L

A slurry of 87.4 mg crystalline sodium cholesteryl sulfate purchased from Avanti Polar Lipids, Inc. in 1 mL of ethanol was magnetically stirred at ambient temperature. After 1 day, the slurry was too thick to stir and an additional 2 mL of EtOH was added. The slurry continued to stir for 13 days at ambient temperature. Crystalline sodium cholesteryl sulfate Form L was harvested by decanting the excess liquor after centrifugation and drying under vacuum at ambient temperature for approximately 2 days. FIGS. 13 and 14 are x-ray powder diffraction patterns of crystalline sodium cholesteryl sulfate Form L from this Example 6.

### Example 7—Preparation of Crystalline Sodium Cholesteryl Sulfate Form M

A slurry of 89.3 mg crystalline sodium cholesteryl sulfate purchased from Avanti Polar Lipids, Inc. in 1 mL of N-methyl-2-pyrrolidone (NMP) was magnetically stirred at ambient temperature for 14 days. Crystalline sodium cholesteryl sulfate Form M was harvested damp by decanting the excess liquor after centrifugation. FIG. 16 is an x-ray powder diffraction pattern of crystalline sodium cholesteryl sulfate Form M from this Example 7, and FIG. 17 is the indexing solution from that x-ray powder diffraction pattern.

### Example 8—Preparation of Crystalline Sodium Cholesteryl Sulfate Form N

Crystalline sodium cholesteryl sulfate purchased from Avanti Polar Lipids, Inc. (39.0 mg) was exposed to water vapor within a closed vessel for 7 days at ambient temperature to provide crystalline sodium cholesteryl sulfate Form N.

### Example 10—Instrumental Techniques

### Differential Scanning Calorimetry (DSC)

DSC was performed using a Mettler-Toledo DSC3+ differential scanning calorimeter. A tau lag adjustment is performed with indium, tin, and zinc. The temperature and enthalpy are adjusted with octane, phenyl salicylate, indium, tin, and zinc. The adjustment is then verified with octane, phenyl salicylate, indium, tin, and zinc. The sample was placed into a hermetically sealed aluminum DSC pan, and the weight was accurately recorded. The pan was then inserted into the DSC cell. A weighed aluminum pan configured as the sample pan was placed on the reference side of the cell. The pan lid was pierced prior to analysis. Samples were analyzed from −30° C. to 250° C. @ 10°/min.

### Dynamic Vapor Sorption/Desorption (DVS)

Automated vapor sorption (VS) data were collected on a Surface Measurement System DVS Intrinsic instrument. Samples were not dried prior to analysis. Sorption and desorption data were collected over a range from 5% to 95% RH at 10% RH increments under a nitrogen purge. The equilibrium criterion used for analysis was less than 0.0100% weight change in 5 minutes with a maximum equilibration time of 3 hours. Data were not corrected for the initial moisture content of the samples.

### Polarized Light Microscopy

Polarized light microscopy was performed using a Motic SMZ-168. Each sample was observed using a 10× objective at 0.75 up to 5.0× magnification with crossed polarizers.

### Proton NMR Spectroscopy

Samples were prepared by dissolving approximately a weighed amount of sample in deuterated solvents and analyzed with one or more commercial spectrometers.

### Thermogravimetric Analysis (TGA)

TG analysis was performed using a Mettler-Toledo TGA/DSC3 analyzer. Temperature and enthalpy adjustments were performed using indium, tin, and zinc, and then verified with indium. The balance was verified with calcium oxalate. The sample was placed in an open aluminum pan. The pan was hermetically sealed, the lid pierced, then inserted into the TG furnace. A weighed aluminum pan configured as the sample pan was placed on the reference platform. The furnace was heated under nitrogen. Each sample was heated from ambient temperature to 350° C. at 10° C./min. Although thermograms are plotted by reference temperature (x-axis), results are reported according to sample temperatures.

### XRPD—Transmission

X-ray powder diffraction pattern was collected with a PANalytical X'Pert PRO MPD or PANalytical Empyrean diffractometer using an incident beam of Cu radiation produced using a long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening and asymmetry from axial divergence. Diffraction patterns were collected using a scanning position-sensitive detector (X'Celerator) located 240 mm from the specimen and Data Collector software v. 5.5. The data acquisition parameters are listed in the image of each pattern displayed in the Data section of this report. All images have the instrument labeled as X'Pert PRO MPD regardless of the instrument used.

### XRPD—Reflection Geometry

X-ray powder diffraction patterns were collected with a PANalytical X'Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 5.5. The data acquisition parameters for each pattern are displayed above the image in the Data section of this report including the divergence slit (DS) and the incident-beam SS.

For x-ray diffractograms showing expanded regions, the y-axis on the left side of the diffractograms reads “4” which the corresponding software has rounded from 3.5°2θ.

1. A non-water solvate of crystalline sodium cholesteryl sulfate.

2. An anhydrate of crystalline sodium cholesteryl sulfate.

3. Crystalline sodium cholesteryl sulfate Form F.

4. Crystalline sodium cholesteryl sulfate of any one of claim 2 or 3, having an x-ray powder diffraction pattern with two peaks between about 1.9°2θ and about 3.3°2θ.

5. Crystalline sodium cholesteryl sulfate of any one of claim 2 or 3-4, having a DSC thermogram with a peak onset of about 217° C.

6. Crystalline sodium cholesteryl sulfate Form L.

7. Crystalline sodium cholesteryl sulfate of any one of claim 2 or 6 having an x-ray powder diffraction pattern comprising three peaks between about 1.9°2θ and about 3.3°2θ.

8. Crystalline sodium cholesteryl sulfate of any one of claim 2 or 7, having a DSC thermogram with a peak onset of about 218° C.

9. Crystalline sodium cholesteryl sulfate Form J.

10. Crystalline sodium cholesteryl sulfate of claim 9, having an x-ray powder diffraction pattern with more than one peak between about 1.5°2θ and about 7.0°2θ.

11. Crystalline sodium cholesteryl sulfate Form N.

12. Crystalline sodium cholesteryl sulfate of claim 11, having an x-ray powder diffraction pattern with a single peak between about 1.5°2θ and about 6.0°2θ.

13. Crystalline sodium cholesteryl sulfate Form H.

14. Crystalline sodium cholesteryl sulfate of any one of claim 2 or 13, having an x-ray powder diffraction pattern comprising a peak at about 2.2°2θ.

15. Crystalline sodium cholesteryl sulfate Form H having an x-ray powder diffraction pattern comprising a peak at about 2.2°2θ and a peak at about 4.8°2θ.

16. Crystalline sodium cholesteryl sulfate Form K.

17. Crystalline sodium cholesteryl sulfate of any one of claim 1 or 16, having an x-ray powder diffraction pattern comprising a peak at about 3.7°2θ.

18. Crystalline sodium cholesteryl sulfate Form M.

19. Crystalline sodium cholesteryl sulfate of any one of claim 1 or 18, having an x-ray powder diffraction pattern comprising a peak at about 3.9°2θ.

20. Crystalline sodium cholesteryl sulfate Form B.

21. A pharmaceutical composition comprising crystalline sodium cholesteryl sulfate.

22. A pharmaceutical composition comprising one or more of crystalline sodium cholesteryl sulfate Form B, crystalline sodium cholesteryl sulfate Form F, crystalline sodium cholesteryl sulfate Form L, crystalline sodium cholesteryl sulfate Form J, crystalline sodium cholesteryl sulfate Form N, crystalline sodium cholesteryl sulfate Form H, crystalline sodium cholesteryl sulfate Form K, and crystalline sodium cholesteryl sulfate Form M and one or more pharmaceutically acceptable excipients.
